SEQUENCE LISTING (1 sequence)

SEQ ID NO: 1 - DNA, 3828 bp, synthetic construct
  source 1..3828: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggccgccaccatgaagattcctaacattggtaatgtgatgaataaatttgagatccttggggttgtaggtgaaggagcctatggagttgtacttaaatgcagacacaaggaaacacatgaaattgtggcgatcaagaaattcaaggacagtgaagaaaatgaagaagtcaaagaaacgactttacgagagcttaaaatgcttcggactctcaagcaggaaaacattgtggagttgaaggaagcatttcgtcggaggggaaagttgtacttggtgtttgagtatgttgaaaaaaatatgctcgaattgctggaagaaatgccaaatggagttccacctgagaaagtaaaaagctacatctatcagctaatcaaggctattcactggtgccataagaatgatattgtccatcgagatataaaaccagaaaatctcttaatcagccacaatgatgtcctaaaactgtgtgactttggttttgctcgtaatctgtcagaaggcaataatgctaattacacagagtacgttgccaccagatggtatcggtccccagaactcttacttggcgctccctatggaaagtccgtggacatgtggtcggtgggctgtattcttggggagcttagcgatggacagcctttatttcctggagaaagtgaaattgaccaactttttactattcagaaggtgctaggaccacttccatctgagcagatgaagcttttctacagtaatcctcgcttccatgggctccggtttccagctgttaaccatcctcagtccttggaaagaagataccttggaattttgaatagtgttctacttgacctaatgaagaatttactgaagttggacccagctgacagatacttgacagaacagtgtttgaatcaccctacatttcaaacccagagacttctggatcgttctccttcaaggtcagcaaaaagaaaaccttaccatgtggaaagcagcacattgtctaatagaaaccaagccggcaaaagtactgctttgcagtctcaccacagatctaacagcaaggacatccagaacctgagtgtaggcctgccccgggctgacgaaggtctccctgccaatgaaagcttcctaaatggaaaccttgctggagctagtcttagtccactgcacaccaaaacctaccaagcaagcagccagcctgggtctaccagcaaagatctcaccaacaacaacataccacaccttcttagcccaaaagaagccaagtcaaaaacagagtttgattttaatattgacccaaagccttcagaaggcccagggacaaagtacctcaagtcaaacagcagatctcagcagaaccgccactcattcatggaaagctctcaaagcaaagctgggacactgcagcccaatgaaaagcagagtcggcatagctatattgacacaattccccagtcctctaggagtccctcctacaggaccaaggccaaaagccatggggcactgagtgactccaagtctgtgagcaacctttctgaagccagggcccaaattgcggagcccagtaccagtaggtacttcccatctagctgcttagacttgaattctcccaccagcccaacccccaccagacacagtgacacgagaactttgctcagcccttctggaagaaataaccgaaatgagggaacgctggactcacgtcgaaccacaaccagacattctaagacgatggaggaattgaagctgccggagcacatggacagtagccattcccattcactgtctgcacctcacgaatctttttcttatggactgggctacaccagccccttttcttcccagcaacgtcctcataggcattctatgtatgtgacccgtgacaaagtgagagccaagggcttggatggaagcttgagcatagggcaagggatggcagctagagccaacagcctgcaactcttgtcaccccagcctggagaacagctccctccagagatgactgtggcaagatcttcggtcaaagagacctccagagaaggcacctcttccttccatacacgccagaagtctgagggtggagtgtatcatgacccacactctgatgatggcacagcccccaaagaaaatagacacctatacaatgatcctgtgccaaggagagttggtagcttttacagagtgccatctccacgtccagacaattctttccatgaaaataatgtgtcaactagagtttcttctctaccatcagagagcagttctggaaccaaccactcaaaaagacaaccagcattcgatccatggaaaagtcctgaaaatattagtcattcagagcaactcaaggaaaaagagaagcaaggatttttcaggtcaatgaaaaagaaaaagaagaaatctcaaacagtacccaattccgacagccctgatcttctgacgttgcagaaatccattcattctgctagcactccaagcagcagaccaaaggagtggcgccccgagaagatctcagatctgcagacccaaagccagccattaaaatcactgcgcaagttgttacatctctcttcggcctcaaatcacccggcttcctcagatccccgcttccagcccttaacagctcaacaaaccaaaaattccttctcagaaattcggattcaccccctgagccaggcctctggcgggagcagcaacatccggcaggaacccgcaccgaagggcaggccagccctccagctgccaggtcagatggatcctggttggcatgtgtcctctgtgaccaggagtgccacagagggcccttcctactctgaacagctgggtgccaaaagtgggccaaatgggcacccctataacagaacaaatcgctcacgaatgccaaatctgaatgatttaaaagagacagccttgtaagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

(19) United States
(12) Patent Application Publication
(10) Pub. No.: US 2026/0306663 A1
(43) Pub. Date: Oct. 8, 2026

(54) FORMULATIONS COMPRISING RECOMBINANT AAV AND METHODS OF ADMINISTERING THE SAME

(71) Applicant: Ultragenyx Pharmaceutical Inc., Novato, CA (US)

(72) Inventors: Heather Lau, Novato, CA (US); Julie Yu Wei, Novato, CA (US); Kirin Jamison, Novato, CA (US); Jan Panteli, Novato, CA (US); Sharyl Lynne Fyffe-Maricich, Novato, CA (US)

(21) Appl. No.: 19/485,158

(22) PCT Filed: May 17, 2024
(86) PCT No.: PCT/US2024/030022

§ 371 Date: Nov. 17, 2025

Related U.S. Application Data
(60) Provisional application No. 63/503,062, filed on May 18, 2023.

(51) Int. Cl. A61K 48/00; A61K 38/45

(52) U.S. Cl. CPC A61K 48/0033; A61K 38/45; A61K 48/005; A61K 48/0075; C12Y 207/11022

(57) ABSTRACT

The present disclosure provides methods for preparing pharmaceutical formulations comprising recombinant adeno-associated virus (rAAV) suitable for administration to the central nervous system (CNS) via intrathecal (IT), intracerebroventricular (ICV), and/or intracisternal magna (ICM) routes. Also described herein are pharmaceutical formulations comprising rAAV suitable for administration to the CNS and methods of their use in the treatment of CNS disorders, e.g., rare genetic CNS disorders such as CDKL5 deficiency disorder (CDD).

### CROSS-REFERENCE TO RELATED APPLICATIONS

This application claims the benefit of and priority to U.S. Provisional Application No. 63/503,062, filed on May 18, 2023, the entire disclosure of which is incorporated by reference herein in its entirety for all purposes.

### SEQUENCE LISTING

This application contains a Sequence Listing which has been submitted electronically in XML format and is hereby incorporated by reference in its entirety. The XML copy, created on Apr. 11, 2024, is named ULTA-031WO_SL.xml and is 5,779 bytes in size.

### TECHNICAL FIELD OF THE DISCLOSURE

The present disclosure relates generally to formulations comprising recombinant adeno-associated virus (rAAV) and methods of their use in gene therapy for the treatment of various genetic disorders.

### BACKGROUND

Development of adeno-associated virus (AAV) gene therapies requires a significant focus on safety and efficacy considerations in the development of a good manufacturing practice (GMP) processes. In developing potential gene therapy treatments for genetic disorders, e.g., rare disorders targeted to the central nervous system (CNS), careful considerations on route of administration, formulation selection, drug product concentration, dosing strategy (including dosing volume and endotoxin limits), and delivery device must all be assessed to ensure patient safety is maintained at an achievable efficacious dose.

For efficacious gene therapy for certain rare disorders, there can be several pre-clinical and clinical challenges. One such disorder is CDKL5 deficiency disorder (CDD), a rare neurodevelopmental disease caused by mutations in the CDKL5 gene which can manifest in a broad range of clinical symptoms and severity. Hallmarks of CDD include infantile-onset refractory epilepsy, developmental delay, intellectual disability, visual impairment, lack of speech, hypotonia, motor dysfunction, sleep disturbances, gastrointestinal dysfunction, and breathing difficulties. Although rare, the occurrence is believed to be approximately 1 in 40,000-60,000 live births, making it one of the most common forms of genetic epilepsy. AAV for the treatment of CDD and formulations comprising the same are described in WO/2021/163322 and WO/2023/023590, respectively, the disclosures of which are herein incorporated by reference in their entireties.

The present disclosure provides methods for preparing pharmaceutical formulations comprising rAAV suitable for administration to the CNS via intrathecal (IT), intracerebroventricular (ICV), and/or intracisternal magna (ICM) routes. Also described herein are pharmaceutical formulations comprising rAAV suitable for administration to the CNS and methods of their use in the treatment of CNS disorders, e.g., rare genetic CNS disorders such as CDD.

### SUMMARY OF THE DISCLOSURE

The present disclosure provides pharmaceutical formulations comprising recombinant adeno-associated virus (rAAV) and methods of their use in gene therapy. Also provided herein are methods for preparing pharmaceutical formulations comprising rAAV suitable for administration to the central nervous system (CNS) via intrathecal (IT), intracerebroventricular (ICV), and/or intracisternal magna (ICM) routes.

In one aspect, the present disclosure provides a pharmaceutical formulation for administration to the CNS of a subject, the pharmaceutical formulation comprising a recombinant adeno-associated virus (rAAV) at a concentration of at least 5×10¹³ genome copies (GC)/mL and having an endotoxin concentration of less than or equal to 0.2 endotoxin units (EU)/mL.

In another aspect, the present disclosure provides methods of treating a central nervous system (CNS) disorder in a subject comprising administering to the CNS of the subject a pharmaceutical formulation comprising a recombinant adeno-associated virus (rAAV) at a concentration of at least 5×10¹³ GC/mL and having an endotoxin concentration of less than or equal to 0.2 EU/mL.

In one embodiment according to the above aspects, the pharmaceutical formulation comprises an rAAV at a concentration of at least 5×10¹³ GC/mL, at least 6×10¹³ GC/mL, at least 7×10¹³ GC/mL, at least 8×10¹³ GC/mL, at least 9×10¹³ GC/mL, at least 1×10¹⁴ GC/mL, at least 1.5×10¹⁴ GC/mL, at least 2×10¹⁴ GC/mL, at least 3×10¹⁴ GC/mL, at least 4×10¹⁴ GC/mL, at least 5×10¹⁴ GC/mL, at least 6×10¹⁴ GC/mL, at least 7×10¹⁴ GC/mL, at least 8×10¹⁴ GC/mL, at least 9×10¹⁴ GC/mL, at least 1×10¹⁵ GC/mL, at least 3×10¹⁵ GC/mL, or at least 5×10¹⁵ GC/mL.

In some embodiments, the pharmaceutical formulation comprises an rAAV at a concentration of at least 5×10¹³ GC/mL. In some embodiments, the pharmaceutical formulation comprises an rAAV at a concentration of at least 6×10¹³ GC/mL. In some embodiments, the pharmaceutical formulation comprises an rAAV at a concentration of at least 7×10¹³ GC/mL. In some embodiments, the pharmaceutical formulation comprises an rAAV at a concentration of at least 8×10¹³ GC/mL. In some embodiments, the pharmaceutical formulation comprises an rAAV at a concentration of at least 9×10¹³ GC/mL. In some embodiments, the pharmaceutical formulation comprises an rAAV at a concentration of at least 1×10¹⁴ GC/mL. In some embodiments, the pharmaceutical formulation comprises an rAAV at a concentration of at least 1.5×10¹⁴ GC/mL. In some embodiments, the pharmaceutical formulation comprises an rAAV at a concentration of at least 2×10¹⁴ GC/mL. In some embodiments, the pharmaceutical formulation comprises an rAAV at a concentration of at least 3×10¹⁴ GC/mL. In some embodiments, the pharmaceutical formulation comprises an rAAV at a concentration of at least 4×10¹⁴ GC/mL. In some embodiments, the pharmaceutical formulation comprises an rAAV at a concentration of at least 5×10¹⁴ GC/mL. In some embodiments, the pharmaceutical formulation comprises an rAAV at a concentration of at least 6×10¹⁴ GC/mL. In some embodiments, the pharmaceutical formulation comprises an rAAV at a concentration of at least 7×10¹⁴ GC/mL. In some embodiments, the pharmaceutical formulation comprises an rAAV at a concentration of at least 8×10¹⁴ GC/mL. In some embodiments, the pharmaceutical formulation comprises an rAAV at a concentration of at least 9×10¹⁴ GC/mL. In some embodiments, the pharmaceutical formulation comprises an rAAV at a concentration of at least 1×10¹⁵ GC/mL. In some embodiments, the pharmaceutical formulation comprises an rAAV at a concentration of at least 3×10¹⁵ GC/mL. In some embodiments, the pharmaceutical formulation comprises an rAAV at a concentration of at least 5×10¹⁵ GC/mL.

In one embodiment according to the above aspects, the pharmaceutical formulation comprises an rAAV at a concentration of 1×10¹⁴ GC/ml to 3×10¹⁵ GC/ml (e.g., 1×10¹⁴ to 2×10¹⁴ GC/ml, 2×10¹⁴ to 3×10¹⁴ GC/ml, 3×10¹⁴ to 4×10¹⁴ GC/ml, 4×10¹⁴ to 5×10¹⁴ GC/ml, 5×10¹⁴ to 6×10¹⁴ GC/ml, 6×10¹⁴ to 7×10¹⁴ GC/ml, 7×10¹⁴ to 8×10¹⁴ GC/ml, 8×10¹⁴ to 9×10¹⁴ GC/ml, 9×10¹⁴ to 1×10¹⁵ GC/ml, 1×10¹⁵ to 2×10¹⁵ GC/ml, 2×10¹⁵ to 3×10¹⁵ GC/ml, 2×10¹⁴ to 2×10¹⁵ GC/ml, 3×10¹⁴ to 1×10¹⁵ GC/ml, 4×10¹⁴ to 9×10¹⁴ GC/ml, 5×10¹⁴ to 8×10¹⁴ GC/ml, and all integers including and in between 1×10¹⁴ to 3×10¹⁵ GC/ml). In some embodiments, the pharmaceutical formulation comprises an rAAV at a concentration of 1×10¹⁴ to 2×10¹⁴ GC/ml. In some embodiments, the pharmaceutical formulation comprises an rAAV at a concentration of 2×10¹⁴ to 3×10¹⁴ GC/ml. In some embodiments, the pharmaceutical formulation comprises an rAAV at a concentration of 3×10¹⁴ to 4×10¹⁴ GC/ml. In some embodiments, the pharmaceutical formulation comprises an rAAV at a concentration of 4×10¹⁴ to 5×10¹⁴ GC/ml. In some embodiments, the pharmaceutical formulation comprises an rAAV at a concentration of 5×10¹⁴ to 6×10¹⁴ GC/ml. In some embodiments, the pharmaceutical formulation comprises an rAAV at a concentration of 6×10¹⁴ to 7×10¹⁴ GC/ml. In some embodiments, the pharmaceutical formulation comprises an rAAV at a concentration of 7×10¹⁴ to 8×10¹⁴ GC/ml. In some embodiments, the pharmaceutical formulation comprises an rAAV at a concentration of 8×10¹⁴ to 9×10¹⁴ GC/ml. In some embodiments, the pharmaceutical formulation comprises an rAAV at a concentration of 9×10¹⁴ to 1×10¹⁵ GC/ml. In some embodiments, the pharmaceutical formulation comprises an rAAV at a concentration of 1×10¹⁵ to 2×10¹⁵ GC/ml. In some embodiments, the pharmaceutical formulation comprises an rAAV at a concentration of 2×10¹⁵ to 3×10¹⁵ GC/ml. In some embodiments, the pharmaceutical formulation comprises an rAAV at a concentration of 2×10¹⁴ to 2×10¹⁵ GC/ml. In some embodiments, the pharmaceutical formulation comprises an rAAV at a concentration of 3×10¹⁴ to 1×10¹⁵ GC/ml. In some embodiments, the pharmaceutical formulation comprises an rAAV at a concentration of 4×10¹⁴ to 9×10¹⁴ GC/ml. In some embodiments, the pharmaceutical formulation comprises an rAAV at a concentration of 5×10¹⁴ to 8×10¹⁴ GC/ml.

In another embodiment according to the above aspects, the pharmaceutical formulation has an endotoxin concentration of less than or equal to 0.2 EU/mL, less than or equal to 0.15 EU/mL, less than or equal to 0.1 EU/mL, less than or equal to 0.05 EU/mL, less than or equal to 0.03 EU/mL, or less than or equal to 0.01 EU/mL. In some embodiments, the pharmaceutical formulation has an endotoxin concentration of less than or equal to 0.2 EU/mL. In some embodiments, the pharmaceutical formulation has an endotoxin concentration of less than or equal to 0.15 EU/mL. In some embodiments, the pharmaceutical formulation has an endotoxin concentration of less than or equal to 0.1 EU/mL. In some embodiments, the pharmaceutical formulation has an endotoxin concentration of less than or equal to 0.05 EU/mL. In some embodiments, the pharmaceutical formulation has an endotoxin concentration of less than or equal to 0.03 EU/mL. In some embodiments, the pharmaceutical formulation has an endotoxin concentration of less than or equal to 0.01 EU/mL.

In yet another embodiment according to the above aspects, the pharmaceutical formulation comprises a recombinant adeno-associated virus (rAAV) at a concentration of at least 5×10¹³ GC/mL and having an endotoxin concentration of less than or equal to 0.2 EU/mL. In some embodiments, the pharmaceutical formulation comprises a recombinant adeno-associated virus (rAAV) at a concentration of at least 5×10¹³ GC/mL and having an endotoxin concentration of less than or equal to 0.15 EU/mL. In some embodiments, the pharmaceutical formulation comprises a recombinant adeno-associated virus (rAAV) at a concentration of at least 5×10¹³ GC/mL and having an endotoxin concentration of less than or equal to 0.1 EU/mL. In some embodiments, the pharmaceutical formulation comprises a recombinant adeno-associated virus (rAAV) at a concentration of at least 5×10¹³ GC/mL and having an endotoxin concentration of less than or equal to 0.05 EU/mL. In some embodiments, the pharmaceutical formulation comprises a recombinant adeno-associated virus (rAAV) at a concentration of at least 5×10¹³ GC/mL and having an endotoxin concentration of less than or equal to 0.03 EU/mL. In some embodiments, the pharmaceutical formulation comprises a recombinant adeno-associated virus (rAAV) at a concentration of at least 5×10¹³ GC/mL and having an endotoxin concentration of less than or equal to 0.01 EU/mL.

In yet another embodiment according to the above aspects, the pharmaceutical formulation comprises a recombinant adeno-associated virus (rAAV) at a concentration of at least 6×10¹³ GC/mL and having an endotoxin concentration of less than or equal to 0.2 EU/mL. In some embodiments, the pharmaceutical formulation comprises a recombinant adeno-associated virus (rAAV) at a concentration of at least 6×10¹³ GC/mL and having an endotoxin concentration of less than or equal to 0.15 EU/mL. In some embodiments, the pharmaceutical formulation comprises a recombinant adeno-associated virus (rAAV) at a concentration of at least 6×10¹³ GC/mL and having an endotoxin concentration of less than or equal to 0.1 EU/mL. In some embodiments, the pharmaceutical formulation comprises a recombinant adeno-associated virus (rAAV) at a concentration of at least 6×10¹³ GC/mL and having an endotoxin concentration of less than or equal to 0.05 EU/mL. In some embodiments, the pharmaceutical formulation comprises a recombinant adeno-associated virus (rAAV) at a concentration of at least 6×10¹³ GC/mL and having an endotoxin concentration of less than or equal to 0.03 EU/mL. In some embodiments, the pharmaceutical formulation comprises a recombinant adeno-associated virus (rAAV) at a concentration of at least 6×10¹³ GC/mL and having an endotoxin concentration of less than or equal to 0.01 EU/mL.

In yet another embodiment according to the above aspects, the pharmaceutical formulation comprises a recombinant adeno-associated virus (rAAV) at a concentration of at least 7×10¹³ GC/mL and having an endotoxin concentration of less than or equal to 0.2 EU/mL. In some embodiments, the pharmaceutical formulation comprises a recombinant adeno-associated virus (rAAV) at a concentration of at least 7×10¹³ GC/mL and having an endotoxin concentration of less than or equal to 0.15 EU/mL. In some embodiments, the pharmaceutical formulation comprises a recombinant adeno-associated virus (rAAV) at a concentration of at least 7×10¹³ GC/mL and having an endotoxin concentration of less than or equal to 0.1 EU/mL. In some embodiments, the pharmaceutical formulation comprises a recombinant adeno-associated virus (rAAV) at a concentration of at least 7×10¹³ GC/mL and having an endotoxin concentration of less than or equal to 0.05 EU/mL. In some embodiments, the pharmaceutical formulation comprises a recombinant adeno-associated virus (rAAV) at a concentration of at least 7×10¹³ GC/mL and having an endotoxin concentration of less than or equal to 0.03 EU/mL. In some embodiments, the pharmaceutical formulation comprises a recombinant adeno-associated virus (rAAV) at a concentration of at least 7×10¹³ GC/mL and having an endotoxin concentration of less than or equal to 0.01 EU/mL.

In yet another embodiment according to the above aspects, the pharmaceutical formulation comprises a recombinant adeno-associated virus (rAAV) at a concentration of at least 8×10¹³ GC/mL and having an endotoxin concentration of less than or equal to 0.2 EU/mL. In some embodiments, the pharmaceutical formulation comprises a recombinant adeno-associated virus (rAAV) at a concentration of at least 8×10¹³ GC/mL and having an endotoxin concentration of less than or equal to 0.15 EU/mL. In some embodiments, the pharmaceutical formulation comprises a recombinant adeno-associated virus (rAAV) at a concentration of at least 8×10¹³ GC/mL and having an endotoxin concentration of less than or equal to 0.1 EU/mL. In some embodiments, the pharmaceutical formulation comprises a recombinant adeno-associated virus (rAAV) at a concentration of at least 8×10¹³ GC/mL and having an endotoxin concentration of less than or equal to 0.05 EU/mL. In some embodiments, the pharmaceutical formulation comprises a recombinant adeno-associated virus (rAAV) at a concentration of at least 8×10¹³ GC/mL and having an endotoxin concentration of less than or equal to 0.03 EU/mL. In some embodiments, the pharmaceutical formulation comprises a recombinant adeno-associated virus (rAAV) at a concentration of at least 8×10¹³ GC/mL and having an endotoxin concentration of less than or equal to 0.01 EU/mL.

In yet another embodiment according to the above aspects, the pharmaceutical formulation comprises a recombinant adeno-associated virus (rAAV) at a concentration of at least 9×10¹³ GC/mL and having an endotoxin concentration of less than or equal to 0.2 EU/mL. In some embodiments, the pharmaceutical formulation comprises a recombinant adeno-associated virus (rAAV) at a concentration of at least 9×10¹³ GC/mL and having an endotoxin concentration of less than or equal to 0.15 EU/mL. In some embodiments, the pharmaceutical formulation comprises a recombinant adeno-associated virus (rAAV) at a concentration of at least 9×10¹³ GC/mL and having an endotoxin concentration of less than or equal to 0.1 EU/mL. In some embodiments, the pharmaceutical formulation comprises a recombinant adeno-associated virus (rAAV) at a concentration of at least 9×10¹³ GC/mL and having an endotoxin concentration of less than or equal to 0.05 EU/mL. In some embodiments, the pharmaceutical formulation comprises a recombinant adeno-associated virus (rAAV) at a concentration of at least 9×10¹³ GC/mL and having an endotoxin concentration of less than or equal to 0.03 EU/mL. In some embodiments, the pharmaceutical formulation comprises a recombinant adeno-associated virus (rAAV) at a concentration of at least 9×10¹³ GC/mL and having an endotoxin concentration of less than or equal to 0.01 EU/mL.

In yet another embodiment according to the above aspects, the pharmaceutical formulation comprises a recombinant adeno-associated virus (rAAV) at a concentration of at least 10×10¹⁴ GC/mL and having an endotoxin concentration of less than or equal to 0.2 EU/mL. In some embodiments, the pharmaceutical formulation comprises a recombinant adeno-associated virus (rAAV) at a concentration of at least 10×10¹⁴ GC/mL and having an endotoxin concentration of less than or equal to 0.15 EU/mL. In some embodiments, the pharmaceutical formulation comprises a recombinant adeno-associated virus (rAAV) at a concentration of at least 10×10¹⁴ GC/mL and having an endotoxin concentration of less than or equal to 0.1 EU/mL. In some embodiments, the pharmaceutical formulation comprises a recombinant adeno-associated virus (rAAV) at a concentration of at least 10×10¹⁴ GC/mL and having an endotoxin concentration of less than or equal to 0.05 EU/mL. In some embodiments, the pharmaceutical formulation comprises a recombinant adeno-associated virus (rAAV) at a concentration of at least 10×10¹⁴ GC/mL and having an endotoxin concentration of less than or equal to 0.03 EU/mL. In some embodiments, the pharmaceutical formulation comprises a recombinant adeno-associated virus (rAAV) at a concentration of at least 10×10¹⁴ GC/mL and having an endotoxin concentration of less than or equal to 0.01 EU/mL.

In yet another embodiment according to the above aspects, the pharmaceutical formulation comprises a recombinant adeno-associated virus (rAAV) at a concentration of at least 1.5×10¹⁴ GC/mL and having an endotoxin concentration of less than or equal to 0.2 EU/mL. In some embodiments, the pharmaceutical formulation comprises a recombinant adeno-associated virus (rAAV) at a concentration of at least 1.5×10¹⁴ GC/mL and having an endotoxin concentration of less than or equal to 0.15 EU/mL. In some embodiments, the pharmaceutical formulation comprises a recombinant adeno-associated virus (rAAV) at a concentration of at least 1.5×10¹⁴ GC/mL and having an endotoxin concentration of less than or equal to 0.1 EU/mL. In some embodiments, the pharmaceutical formulation comprises a recombinant adeno-associated virus (rAAV) at a concentration of at least 1.5×10¹⁴ GC/mL and having an endotoxin concentration of less than or equal to 0.05 EU/mL. In some embodiments, the pharmaceutical formulation comprises a recombinant adeno-associated virus (rAAV) at a concentration of at least 1.5×10¹⁴ GC/mL and having an endotoxin concentration of less than or equal to 0.03 EU/mL. In some embodiments, the pharmaceutical formulation comprises a recombinant adeno-associated virus (rAAV) at a concentration of at least 1.5×10¹⁴ GC/mL and having an endotoxin concentration of less than or equal to 0.01 EU/mL.

In yet another embodiment according to the above aspects, the pharmaceutical formulation comprises a recombinant adeno-associated virus (rAAV) at a concentration of at least 2×10¹⁴ GC/mL and having an endotoxin concentration of less than or equal to 0.2 EU/mL. In some embodiments, the pharmaceutical formulation comprises a recombinant adeno-associated virus (rAAV) at a concentration of at least 2×10¹⁴ GC/mL and having an endotoxin concentration of less than or equal to 0.15 EU/mL. In some embodiments, the pharmaceutical formulation comprises a recombinant adeno-associated virus (rAAV) at a concentration of at least 2×10¹⁴ GC/mL and having an endotoxin concentration of less than or equal to 0.1 EU/mL. In some embodiments, the pharmaceutical formulation comprises a recombinant adeno-associated virus (rAAV) at a concentration of at least 2×10¹⁴ GC/mL and having an endotoxin concentration of less than or equal to 0.05 EU/mL. In some embodiments, the pharmaceutical formulation comprises a recombinant adeno-associated virus (rAAV) at a concentration of at least 2×10¹⁴ GC/mL and having an endotoxin concentration of less than or equal to 0.03 EU/mL. In some embodiments, the pharmaceutical formulation comprises a recombinant adeno-associated virus (rAAV) at a concentration of at least 2×10¹⁴ GC/mL and having an endotoxin concentration of less than or equal to 0.01 EU/mL.

In yet another embodiment according to the above aspects, the pharmaceutical formulation comprises a recombinant adeno-associated virus (rAAV) at a concentration of at least 3×10¹⁴ GC/mL and having an endotoxin concentration of less than or equal to 0.2 EU/mL. In some embodiments, the pharmaceutical formulation comprises a recombinant adeno-associated virus (rAAV) at a concentration of at least 3×10¹⁴ GC/mL and having an endotoxin concentration of less than or equal to 0.15 EU/mL. In some embodiments, the pharmaceutical formulation comprises a recombinant adeno-associated virus (rAAV) at a concentration of at least 3×10¹⁴ GC/mL and having an endotoxin concentration of less than or equal to 0.1 EU/mL. In some embodiments, the pharmaceutical formulation comprises a recombinant adeno-associated virus (rAAV) at a concentration of at least 3×10¹⁴ GC/mL and having an endotoxin concentration of less than or equal to 0.05 EU/mL. In some embodiments, the pharmaceutical formulation comprises a recombinant adeno-associated virus (rAAV) at a concentration of at least 3×10¹⁴ GC/mL and having an endotoxin concentration of less than or equal to 0.03 EU/mL. In some embodiments, the pharmaceutical formulation comprises a recombinant adeno-associated virus (rAAV) at a concentration of at least 3×10¹⁴ GC/mL and having an endotoxin concentration of less than or equal to 0.01 EU/mL.

In yet another embodiment according to the above aspects, the pharmaceutical formulation comprises a recombinant adeno-associated virus (rAAV) at a concentration of at least 4×10¹⁴ GC/mL and having an endotoxin concentration of less than or equal to 0.2 EU/mL. In some embodiments, the pharmaceutical formulation comprises a recombinant adeno-associated virus (rAAV) at a concentration of at least 4×10¹⁴ GC/mL and having an endotoxin concentration of less than or equal to 0.15 EU/mL. In some embodiments, the pharmaceutical formulation comprises a recombinant adeno-associated virus (rAAV) at a concentration of at least 4×10¹⁴ GC/mL and having an endotoxin concentration of less than or equal to 0.1 EU/mL. In some embodiments, the pharmaceutical formulation comprises a recombinant adeno-associated virus (rAAV) at a concentration of at least 4×10¹⁴ GC/mL and having an endotoxin concentration of less than or equal to 0.05 EU/mL. In some embodiments, the pharmaceutical formulation comprises a recombinant adeno-associated virus (rAAV) at a concentration of at least 4×10¹⁴ GC/mL and having an endotoxin concentration of less than or equal to 0.03 EU/mL. In some embodiments, the pharmaceutical formulation comprises a recombinant adeno-associated virus (rAAV) at a concentration of at least 4×10¹⁴ GC/mL and having an endotoxin concentration of less than or equal to 0.01 EU/mL.

In yet another embodiment according to the above aspects, the pharmaceutical formulation comprises a recombinant adeno-associated virus (rAAV) at a concentration of at least 5×10¹⁴ GC/mL and having an endotoxin concentration of less than or equal to 0.2 EU/mL. In some embodiments, the pharmaceutical formulation comprises a recombinant adeno-associated virus (rAAV) at a concentration of at least 5×10¹⁴ GC/mL and having an endotoxin concentration of less than or equal to 0.15 EU/mL. In some embodiments, the pharmaceutical formulation comprises a recombinant adeno-associated virus (rAAV) at a concentration of at least 5×10¹⁴ GC/mL and having an endotoxin concentration of less than or equal to 0.1 EU/mL. In some embodiments, the pharmaceutical formulation comprises a recombinant adeno-associated virus (rAAV) at a concentration of at least 5×10¹⁴ GC/mL and having an endotoxin concentration of less than or equal to 0.05 EU/mL. In some embodiments, the pharmaceutical formulation comprises a recombinant adeno-associated virus (rAAV) at a concentration of at least 5×10¹⁴ GC/mL and having an endotoxin concentration of less than or equal to 0.03 EU/mL. In some embodiments, the pharmaceutical formulation comprises a recombinant adeno-associated virus (rAAV) at a concentration of at least 5×10¹⁴ GC/mL and having an endotoxin concentration of less than or equal to 0.01 EU/mL.

In yet another embodiment according to the above aspects, the pharmaceutical formulation comprises a recombinant adeno-associated virus (rAAV) at a concentration of at least 6×10¹⁴ GC/mL and having an endotoxin concentration of less than or equal to 0.2 EU/mL. In some embodiments, the pharmaceutical formulation comprises a recombinant adeno-associated virus (rAAV) at a concentration of at least 6×10¹⁴ GC/mL and having an endotoxin concentration of less than or equal to 0.15 EU/mL. In some embodiments, the pharmaceutical formulation comprises a recombinant adeno-associated virus (rAAV) at a concentration of at least 6×10¹⁴ GC/mL and having an endotoxin concentration of less than or equal to 0.1 EU/mL. In some embodiments, the pharmaceutical formulation comprises a recombinant adeno-associated virus (rAAV) at a concentration of at least 6×10¹⁴ GC/mL and having an endotoxin concentration of less than or equal to 0.05 EU/mL. In some embodiments, the pharmaceutical formulation comprises a recombinant adeno-associated virus (rAAV) at a concentration of at least 6×10¹⁴ GC/mL and having an endotoxin concentration of less than or equal to 0.03 EU/mL. In some embodiments, the pharmaceutical formulation comprises a recombinant adeno-associated virus (rAAV) at a concentration of at least 6×10¹⁴ GC/mL and having an endotoxin concentration of less than or equal to 0.01 EU/mL.

In yet another embodiment according to the above aspects, the pharmaceutical formulation comprises a recombinant adeno-associated virus (rAAV) at a concentration of at least 7×10¹⁴ GC/mL and having an endotoxin concentration of less than or equal to 0.2 EU/mL. In some embodiments, the pharmaceutical formulation comprises a recombinant adeno-associated virus (rAAV) at a concentration of at least 7×10¹⁴ GC/mL and having an endotoxin concentration of less than or equal to 0.15 EU/mL. In some embodiments, the pharmaceutical formulation comprises a recombinant adeno-associated virus (rAAV) at a concentration of at least 7×10¹⁴ GC/mL and having an endotoxin concentration of less than or equal to 0.1 EU/mL. In some embodiments, the pharmaceutical formulation comprises a recombinant adeno-associated virus (rAAV) at a concentration of at least 7×10¹⁴ GC/mL and having an endotoxin concentration of less than or equal to 0.05 EU/mL. In some embodiments, the pharmaceutical formulation comprises a recombinant adeno-associated virus (rAAV) at a concentration of at least 7×10¹⁴ GC/mL and having an endotoxin concentration of less than or equal to 0.03 EU/mL. In some embodiments, the pharmaceutical formulation comprises a recombinant adeno-associated virus (rAAV) at a concentration of at least 7×10¹⁴ GC/mL and having an endotoxin concentration of less than or equal to 0.01 EU/mL.

In yet another embodiment according to the above aspects, the pharmaceutical formulation comprises a recombinant adeno-associated virus (rAAV) at a concentration of at least 8×10¹⁴ GC/mL and having an endotoxin concentration of less than or equal to 0.2 EU/mL. In some embodiments, the pharmaceutical formulation comprises a recombinant adeno-associated virus (rAAV) at a concentration of at least 8×10¹⁴ GC/mL and having an endotoxin concentration of less than or equal to 0.15 EU/mL. In some embodiments, the pharmaceutical formulation comprises a recombinant adeno-associated virus (rAAV) at a concentration of at least 8×10¹⁴ GC/mL and having an endotoxin concentration of less than or equal to 0.1 EU/mL. In some embodiments, the pharmaceutical formulation comprises a recombinant adeno-associated virus (rAAV) at a concentration of at least 8×10¹⁴ GC/mL and having an endotoxin concentration of less than or equal to 0.05 EU/mL. In some embodiments, the pharmaceutical formulation comprises a recombinant adeno-associated virus (rAAV) at a concentration of at least 8×10¹⁴ GC/mL and having an endotoxin concentration of less than or equal to 0.03 EU/mL. In some embodiments, the pharmaceutical formulation comprises a recombinant adeno-associated virus (rAAV) at a concentration of at least 8×10¹⁴ GC/mL and having an endotoxin concentration of less than or equal to 0.01 EU/mL.

In yet another embodiment according to the above aspects, the pharmaceutical formulation comprises a recombinant adeno-associated virus (rAAV) at a concentration of at least 9×10¹⁴ GC/mL and having an endotoxin concentration of less than or equal to 0.2 EU/mL. In some embodiments, the pharmaceutical formulation comprises a recombinant adeno-associated virus (rAAV) at a concentration of at least 9×10¹⁴ GC/mL and having an endotoxin concentration of less than or equal to 0.15 EU/mL. In some embodiments, the pharmaceutical formulation comprises a recombinant adeno-associated virus (rAAV) at a concentration of at least 9×10¹⁴ GC/mL and having an endotoxin concentration of less than or equal to 0.1 EU/mL. In some embodiments, the pharmaceutical formulation comprises a recombinant adeno-associated virus (rAAV) at a concentration of at least 9×10¹⁴ GC/mL and having an endotoxin concentration of less than or equal to 0.05 EU/mL. In some embodiments, the pharmaceutical formulation comprises a recombinant adeno-associated virus (rAAV) at a concentration of at least 9×10¹⁴ GC/mL and having an endotoxin concentration of less than or equal to 0.03 EU/mL. In some embodiments, the pharmaceutical formulation comprises a recombinant adeno-associated virus (rAAV) at a concentration of at least 9×10¹⁴ GC/mL and having an endotoxin concentration of less than or equal to 0.01 EU/mL.

In yet another embodiment according to the above aspects, the pharmaceutical formulation comprises a recombinant adeno-associated virus (rAAV) at a concentration of at least 1×10¹⁵ GC/mL and having an endotoxin concentration of less than or equal to 0.2 EU/mL. In some embodiments, the pharmaceutical formulation comprises a recombinant adeno-associated virus (rAAV) at a concentration of at least 1×10¹⁵ GC/mL and having an endotoxin concentration of less than or equal to 0.15 EU/mL. In some embodiments, the pharmaceutical formulation comprises a recombinant adeno-associated virus (rAAV) at a concentration of at least 1×10¹⁵ GC/mL and having an endotoxin concentration of less than or equal to 0.1 EU/mL. In some embodiments, the pharmaceutical formulation comprises a recombinant adeno-associated virus (rAAV) at a concentration of at least 1×10¹⁵ GC/mL and having an endotoxin concentration of less than or equal to 0.05 EU/mL. In some embodiments, the pharmaceutical formulation comprises a recombinant adeno-associated virus (rAAV) at a concentration of at least 1×10¹⁵ GC/mL and having an endotoxin concentration of less than or equal to 0.03 EU/mL. In some embodiments, the pharmaceutical formulation comprises a recombinant adeno-associated virus (rAAV) at a concentration of at least 1×10¹⁵ GC/mL and having an endotoxin concentration of less than or equal to 0.01 EU/mL.

In yet another embodiment according to the above aspects, the pharmaceutical formulation comprises a recombinant adeno-associated virus (rAAV) at a concentration of at least 3×10¹⁵ GC/mL and having an endotoxin concentration of less than or equal to 0.2 EU/mL. In some embodiments, the pharmaceutical formulation comprises a recombinant adeno-associated virus (rAAV) at a concentration of at least 3×10¹⁵ GC/mL and having an endotoxin concentration of less than or equal to 0.15 EU/mL. In some embodiments, the pharmaceutical formulation comprises a recombinant adeno-associated virus (rAAV) at a concentration of at least 3×10¹⁵ GC/mL and having an endotoxin concentration of less than or equal to 0.1 EU/mL. In some embodiments, the pharmaceutical formulation comprises a recombinant adeno-associated virus (rAAV) at a concentration of at least 3×10¹⁵ GC/mL and having an endotoxin concentration of less than or equal to 0.05 EU/mL. In some embodiments, the pharmaceutical formulation comprises a recombinant adeno-associated virus (rAAV) at a concentration of at least 3×10¹⁵ GC/mL and having an endotoxin concentration of less than or equal to 0.03 EU/mL. In some embodiments, the pharmaceutical formulation comprises a recombinant adeno-associated virus (rAAV) at a concentration of at least 3×10¹⁵ GC/mL and having an endotoxin concentration of less than or equal to 0.01 EU/mL.

In yet another embodiment according to the above aspects, the pharmaceutical formulation comprises a recombinant adeno-associated virus (rAAV) at a concentration of at least 5×10¹⁵ GC/mL and having an endotoxin concentration of less than or equal to 0.2 EU/mL. In some embodiments, the pharmaceutical formulation comprises a recombinant adeno-associated virus (rAAV) at a concentration of at least 5×10¹⁵ GC/mL and having an endotoxin concentration of less than or equal to 0.15 EU/mL. In some embodiments, the pharmaceutical formulation comprises a recombinant adeno-associated virus (rAAV) at a concentration of at least 5×10¹⁵ GC/mL and having an endotoxin concentration of less than or equal to 0.1 EU/mL. In some embodiments, the pharmaceutical formulation comprises a recombinant adeno-associated virus (rAAV) at a concentration of at least 5×10¹⁵ GC/mL and having an endotoxin concentration of less than or equal to 0.05 EU/mL. In some embodiments, the pharmaceutical formulation comprises a recombinant adeno-associated virus (rAAV) at a concentration of at least 5×10¹⁵ GC/mL and having an endotoxin concentration of less than or equal to 0.03 EU/mL. In some embodiments, the pharmaceutical formulation comprises a recombinant adeno-associated virus (rAAV) at a concentration of at least 5×10¹⁵ GC/mL and having an endotoxin concentration of less than or equal to 0.01 EU/mL.

In one embodiment according to the above aspects, the pharmaceutical formulation comprises an rAAV at a concentration of 1×10¹⁴ GC/ml in the pharmaceutical formulation. In one embodiment according to the above aspects, the pharmaceutical formulation comprises an rAAV at a concentration of 1.5×10¹⁴ GC/ml in the pharmaceutical formulation. In one embodiment according to the above aspects, the pharmaceutical formulation comprises an rAAV at a concentration of 2×10¹⁴ GC/ml in the pharmaceutical formulation.

In yet another embodiment according to the above aspects, the pharmaceutical formulation comprises a recombinant adeno-associated virus (rAAV) at a concentration of at least 1×10¹⁴ GC/mL and having an endotoxin concentration of less than or equal to 0.1 EU/mL. In yet another embodiment according to the above aspects, the pharmaceutical formulation comprises a recombinant adeno-associated virus (rAAV) at a concentration of at least 1.5×10¹⁴ GC/mL and having an endotoxin concentration of less than or equal to 0.1 EU/mL. In yet another embodiment according to the above aspects, the pharmaceutical formulation comprises a recombinant adeno-associated virus (rAAV) at a concentration of at least 2×10¹⁴ GC/mL and having an endotoxin concentration of less than or equal to 0.2 EU/mL.

In yet another embodiment according to the above aspects, the pharmaceutical formulation comprises a total rAAV dose of 3.6×10¹⁴ GC to 3×10¹⁵ GC (e.g., 3.6×10¹⁴ to 4×10¹⁴ GC, 4×10¹⁴ to 5×10¹⁴ GC, 5×10¹⁴ to 6×10¹⁴ GC, 6×10¹⁴ to 7×10¹⁴ GC, 7×10¹⁴ to 8×10¹⁴ GC, 8×10¹⁴ to 9×10¹⁴ GC, 9×10¹⁴ to 1×10¹⁵ GC, 1×10¹⁵ to 2×10¹⁵ GC, 2×10¹⁵ to 3×10¹⁵ GC, 4×10¹⁵ to 2×10¹⁵ GC, 5×10¹⁴ to 1×10¹⁵ GC, 6×10¹⁴ to 9×10¹⁴ GC, and all integers including and in between 3.6×10¹⁴ to 3×10¹⁵ GC). In some embodiments, the pharmaceutical formulation comprises a total rAAV dose of 3.6×10¹⁴ to 4×10¹⁴ GC. In some embodiments, the pharmaceutical formulation comprises a total rAAV dose of 4×10¹⁴ to 5×10¹⁴ GC. In some embodiments, the pharmaceutical formulation comprises a total rAAV dose of 5×10¹⁴ to 6×10¹⁴ GC. In some embodiments, the pharmaceutical formulation comprises a total rAAV dose of 6×10¹⁴ to 7×10¹⁴ GC. In some embodiments, the pharmaceutical formulation comprises a total rAAV dose of 7×10¹⁴ to 8×10¹⁴ GC. In some embodiments, the pharmaceutical formulation comprises a total rAAV dose of 8×10¹⁴ to 9×10¹⁴ GC. In some embodiments, the pharmaceutical formulation comprises a total rAAV dose of 9×10¹⁴ to 1×10¹⁵ GC. In some embodiments, the pharmaceutical formulation comprises a total rAAV dose of 1×10¹⁵ to 2×10¹⁵ GC. In some embodiments, the pharmaceutical formulation comprises a total rAAV dose of 2×10¹⁵ to 3×10¹⁵ GC. In some embodiments, the pharmaceutical formulation comprises a total rAAV dose of 4×10¹⁵ to 2×10¹⁵ GC. In some embodiments, the pharmaceutical formulation comprises a total rAAV dose of 5×10¹⁴ to 1×10¹⁵ GC. In some embodiments, the pharmaceutical formulation comprises a total rAAV dose of 6×10¹⁴ to 9×10¹⁴ GC.

In yet another embodiment according to the above aspects, the pharmaceutical formulation is dosed at 0.1×10¹² GC/g brain to 3×10¹² GC/g brain (e.g., 0.1×10¹² to 0.5×10¹² GC/g brain, 0.5×10¹² to 1×10¹² GC/g brain, 1×10¹² to 1.5×10¹² GC/g brain, 1.5×10¹² to 2×10¹² GC/g brain, 2×10¹² to 2.5×10¹² GC/g brain, 2.5×10¹² to 3×10¹² GC/g brain, 0.5×10¹² to 2.5×10¹² GC/g brain, 1×10¹² to 2×10¹² GC/g brain, and all integers including and in between 0.1×10¹² GC/g brain to 3×10¹² GC/g brain) of the subject. In some embodiments, the pharmaceutical formulation is dosed at 0.1×10¹² GC/g brain to 3×10¹² GC/g brain. In some embodiments, the pharmaceutical formulation is dosed at 0.1×10¹² to 0.5×10¹² GC/g brain. In some embodiments, the pharmaceutical formulation is dosed at 0.5×10¹² to 1×10¹² GC/g brain. In some embodiments, the pharmaceutical formulation is dosed at 1×10¹² to 1.5×10¹² GC/g brain. In some embodiments, the pharmaceutical formulation is dosed at 1.5×10¹² to 2×10¹² GC/g brain. In some embodiments, the pharmaceutical formulation is dosed at 2×10¹² to 2.5×10¹² GC/g brain. In some embodiments, the pharmaceutical formulation is dosed at 2.5×10¹² to 3×10¹² GC/g brain. In some embodiments, the pharmaceutical formulation is dosed at 0.5×10¹² to 2.5×10¹² GC/g brain. In some embodiments, the pharmaceutical formulation is dosed at 1×10¹² to 2×10¹² GC/g brain. In some embodiments, the pharmaceutical formulation is dosed at 1×10¹² GC/g brain of the subject.

In yet another embodiment according to the above aspects, the pharmaceutical formulation comprises sodium phosphate, sodium chloride (NaCl), sorbitol, poloxamer 188, potassium chloride (KCl), magnesium chloride (MgCl₂), calcium chloride (CaCl2), or a combination thereof.

In some embodiments, the pharmaceutical formulation comprises sorbitol at a concentration of at least 1% to at least 20% (e.g., at least 1% to at least 1.5%, at least 1.5% to at least 2%, at least 2% to at least 2.5%, at least 2.5% to at least 3%, at least 3% to at least 3.5%, at least 3.5% to at least 4%, at least 4% to at least 4.5%, at least 4.5% to at least 5%, at least 5% to at least 5.5%, at least 5.5% to at least 6%, at least 6% to at least 6.5%, at least 6.5% to at least 7%, at least 7% to at least 7.5%, at least 7.5% to at least 8%, at least 8% to at least 8.5%, at least 8.5% to at least 9%, at least 9% to at least 9.5%, at least 9.5% to at least 10%, at least 10% to at least 10.5%, at least 10.5% to at least 11%, at least 11% to at least 11.5%, at least 11.5% to at least 12%, at least 12% to at least 12.5%, at least 12.5% to at least 13%, at least 13% to at least 13.5%, at least 13.5% to at least 14%, at least 14% to at least 14.5%, at least 14.5% to at least 15%, at least 15% to at least 15.5%, at least 15.5% to at least 16%, at least 16% to at least 16.5%, at least 16.5% to at least 17%, at least 17% to at least 17.5%, at least 17.5% to at least 18%, at least 18% to at least 18.5%, at least 18.5% to at least 19%, at least 19% to at least 19.5%, at least 19.5% to at least 20, at least 1.5% to at least 19.5%, at least 2% to at least 19%, at least 2.5% to at least 18.5%, at least 3% to at least 18%, at least 3.5% to at least 17.5%, at least 4% to at least 17%, at least 4.5% to at least 16.5%, at least 5% to at least 16%, at least 5.5% to at least 15.5%, at least 6% to at least 15%, at least 7% to at least 14.5%, at least 7.5% to at least 14%, at least 8% to at least 13.5%, at least 8.5% to at least 13%, at least 9% to at least 12.5%, at least 9.5% to at least 12%, at least 10% to at least 11.5%, and all percentages including and in between at least 1% to at least 20%) of the total formulation. In some embodiments, the pharmaceutical formulation comprises sorbitol at a concentration of at least 1% to at least 20% of the total formulation. In some embodiments, the pharmaceutical formulation comprises sorbitol at a concentration of at least 1% to at least 1.5% of the total formulation. In some embodiments, the pharmaceutical formulation comprises sorbitol at a concentration of at least 1.5% to at least 2% of the total formulation. In some embodiments, the pharmaceutical formulation comprises sorbitol at a concentration of at least 2% to at least 2.5% of the total formulation. In some embodiments, the pharmaceutical formulation comprises sorbitol at a concentration of at least 2.5% to at least 3% of the total formulation. In some embodiments, the pharmaceutical formulation comprises sorbitol at a concentration of at least 3% to at least 3.5% of the total formulation. In some embodiments, the pharmaceutical formulation comprises sorbitol at a concentration of at least 3.5% to at least 4% of the total formulation. In some embodiments, the pharmaceutical formulation comprises sorbitol at a concentration of at least 4% to at least 4.5% of the total formulation. In some embodiments, the pharmaceutical formulation comprises sorbitol at a concentration of at least 4.5% to at least 5% of the total formulation. In some embodiments, the pharmaceutical formulation comprises sorbitol at a concentration of at least 5% to at least 5.5% of the total formulation. In some embodiments, the pharmaceutical formulation comprises sorbitol at a concentration of at least 5.5% to at least 6% of the total formulation. In some embodiments, the pharmaceutical formulation comprises sorbitol at a concentration of at least 6% to at least 6.5% of the total formulation. In some embodiments, the pharmaceutical formulation comprises sorbitol at a concentration of at least 6.5% to at least 7% of the total formulation. In some embodiments, the pharmaceutical formulation comprises sorbitol at a concentration of at least 7% to at least 7.5% of the total formulation. In some embodiments, the pharmaceutical formulation comprises sorbitol at a concentration of at least 7.5% to at least 8% of the total formulation. In some embodiments, the pharmaceutical formulation comprises sorbitol at a concentration of at least 8% to at least 8.5% of the total formulation. In some embodiments, the pharmaceutical formulation comprises sorbitol at a concentration of at least 8.5% to at least 9% of the total formulation. In some embodiments, the pharmaceutical formulation comprises sorbitol at a concentration of at least 9% to at least 9.5% of the total formulation. In some embodiments, the pharmaceutical formulation comprises sorbitol at a concentration of at least 9.5% to at least 10% of the total formulation. In some embodiments, the pharmaceutical formulation comprises sorbitol at a concentration of at least 10% to at least 10.5% of the total formulation. In some embodiments, the pharmaceutical formulation comprises sorbitol at a concentration of at least 10.5% to at least 11% of the total formulation. In some embodiments, the pharmaceutical formulation comprises sorbitol at a concentration of at least 11% to at least 11.5% of the total formulation. In some embodiments, the pharmaceutical formulation comprises sorbitol at a concentration of at least 11.5% to at least 12% of the total formulation. In some embodiments, the pharmaceutical formulation comprises sorbitol at a concentration of at least 12% to at least 12.5% of the total formulation. In some embodiments, the pharmaceutical formulation comprises sorbitol at a concentration of at least 12.5% to at least 13% of the total formulation. In some embodiments, the pharmaceutical formulation comprises sorbitol at a concentration of at least 13% to at least 13.5% of the total formulation. In some embodiments, the pharmaceutical formulation comprises sorbitol at a concentration of at least 13.5% to at least 14% of the total formulation. In some embodiments, the pharmaceutical formulation comprises sorbitol at a concentration of at least 14% to at least 14.5% of the total formulation. In some embodiments, the pharmaceutical formulation comprises sorbitol at a concentration of at least 14.5% to at least 15% of the total formulation. In some embodiments, the pharmaceutical formulation comprises sorbitol at a concentration of at least 15% to at least 15.5% of the total formulation. In some embodiments, the pharmaceutical formulation comprises sorbitol at a concentration of at least 15.5% to at least 16% of the total formulation. In some embodiments, the pharmaceutical formulation comprises sorbitol at a concentration of at least 16% to at least 16.5% of the total formulation. In some embodiments, the pharmaceutical formulation comprises sorbitol at a concentration of at least 16.5% to at least 17% of the total formulation. In some embodiments, the pharmaceutical formulation comprises sorbitol at a concentration of at least 17% to at least 17.5% of the total formulation. In some embodiments, the pharmaceutical formulation comprises sorbitol at a concentration of at least 17.5% to at least 18% of the total formulation. In some embodiments, the pharmaceutical formulation comprises sorbitol at a concentration of at least 18% to at least 18.5% of the total formulation. In some embodiments, the pharmaceutical formulation comprises sorbitol at a concentration of at least 18.5% to at least 19% of the total formulation. In some embodiments, the pharmaceutical formulation comprises sorbitol at a concentration of at least 19% to at least 19.5% of the total formulation. In some embodiments, the pharmaceutical formulation comprises sorbitol at a concentration of at least 19.5% to at least 20% of the total formulation. In some embodiments, the pharmaceutical formulation comprises sorbitol at a concentration of at least 1.5% to at least 19.5% of the total formulation. In some embodiments, the pharmaceutical formulation comprises sorbitol at a concentration of at least 2% to at least 19% of the total formulation. In some embodiments, the pharmaceutical formulation comprises sorbitol at a concentration of at least 2.5% to at least 18.5% of the total formulation. In some embodiments, the pharmaceutical formulation comprises sorbitol at a concentration of at least 3% to at least 18% of the total formulation. In some embodiments, the pharmaceutical formulation comprises sorbitol at a concentration of at least 3.5% to at least 17.5% of the total formulation. In some embodiments, the pharmaceutical formulation comprises sorbitol at a concentration of at least 4% to at least 17% of the total formulation. In some embodiments, the pharmaceutical formulation comprises sorbitol at a concentration of at least 4.5% to at least 16.5% of the total formulation. In some embodiments, the pharmaceutical formulation comprises sorbitol at a concentration of at least 5% to at least 16% of the total formulation. In some embodiments, the pharmaceutical formulation comprises sorbitol at a concentration of at least 5.5% to at least 15.5% of the total formulation. In some embodiments, the pharmaceutical formulation comprises sorbitol at a concentration of at least 6% to at least 15% of the total formulation. In some embodiments, the pharmaceutical formulation comprises sorbitol at a concentration of at least 7% to at least 14.5% of the total formulation. In some embodiments, the pharmaceutical formulation comprises sorbitol at a concentration of at least 7.5% to at least 14% of the total formulation. In some embodiments, the pharmaceutical formulation comprises sorbitol at a concentration of at least 8% to at least 13.5% of the total formulation. In some embodiments, the pharmaceutical formulation comprises sorbitol at a concentration of at least 8.5% to at least 13% of the total formulation. In some embodiments, the pharmaceutical formulation comprises sorbitol at a concentration of at least 9% to at least 12.5% of the total formulation. In some embodiments, the pharmaceutical formulation comprises sorbitol at a concentration of at least 9.5% to at least 12% of the total formulation. In some embodiments, the pharmaceutical formulation comprises sorbitol at a concentration of at least 10% to at least 11.5% of the total formulation. In some embodiments, the pharmaceutical formulation comprises sorbitol at a concentration of at least 5% to at least 10% of the total volume of the formulation. In some embodiments, the pharmaceutical formulation comprises sorbitol at a concentration of at least 5% of the total volume of the formulation.

In yet another embodiment according to the above aspects, the pharmaceutical formulation comprises: (i) 10 mM sodium phosphate, 135 mM NaCl, 0.27M Sorbitol, 0.001% v/v poloxamer 188, pH 7.2; (ii) 10 mM sodium phosphate, 135 mM NaCl, 0.54M Sorbitol, 0.001% v/v poloxamer 188, pH 7.2; or (iii) 10 mM Tris, 130 mM NaCl, 2 mM KCl, 1 mM MgCl₂, 2 mM CaCl₂), 0.27M Sorbitol, 0.001% v/v poloxamer 188, pH 6.8. In some embodiments, the pharmaceutical formulation comprises 10 mM sodium phosphate, 135 mM NaCl, 0.27M Sorbitol, 0.001% v/v poloxamer 188, pH 7.2.

In yet another embodiment according to the above aspects, the pharmaceutical formulation is administered intrathecally, intracerebroventricularly, or via an intracisternal magna route. The administration may be a bolus injection or it may be a continuous infusion.

In yet another embodiment according to the above aspects, the pharmaceutical formulation is provided in a vial holding 1 mL to 20 mL (e.g., 1 mL to 4 mL, 4 mL to 8 mL, 8 mL to 12 mL, 12 mL to 16 mL, 16 mL to 20 mL, 4 mL to 16 mL, 8 mL to 16 mL, 12 mL to 20 mL, and all integers including and in between 1 mL to 20 mL). In some embodiments, the pharmaceutical formulation is provided in a vial holding 1 mL to 20 mL. In some embodiments, the pharmaceutical formulation is provided in a vial holding 1 mL to 4 mL. In some embodiments, the pharmaceutical formulation is provided in a vial holding 4 mL to 8 mL. In some embodiments, the pharmaceutical formulation is provided in a vial holding 8 mL to 12 mL. In some embodiments, the pharmaceutical formulation is provided in a vial holding 12 mL to 16 mL. In some embodiments, the pharmaceutical formulation is provided in a vial holding 16 mL to 20 mL. In some embodiments, the pharmaceutical formulation is provided in a vial holding 4 mL to 16 mL. In some embodiments, the pharmaceutical formulation is provided in a vial holding 8 mL to 16 mL. In some embodiments, the pharmaceutical formulation is provided in a vial holding 12 mL to 20 mL. In one embodiment, the pharmaceutical formulation is provided in a vial holding 1 mL, 2 mL, 3 mL, 4 mL, 5 mL, 6 mL, 7 mL, 8 mL, 9 mL, or 10 mL. In some embodiments, the pharmaceutical formulation is provided in a vial holding 1 mL. In an exemplary embodiment, the pharmaceutical formulation is provided in a vial holding 2 mL. In some embodiments, the pharmaceutical formulation is provided in a vial holding 3 mL. In some embodiments, the pharmaceutical formulation is provided in a vial holding 4 mL. In some embodiments, the pharmaceutical formulation is provided in a vial holding 5 mL. In some embodiments, the pharmaceutical formulation is provided in a vial holding 6 mL. In some embodiments, the pharmaceutical formulation is provided in a vial holding 7 mL. In some embodiments, the pharmaceutical formulation is provided in a vial holding 8 mL. In some embodiments, the pharmaceutical formulation is provided in a vial holding 9 mL. In some embodiments, the pharmaceutical formulation is provided in a vial holding 10 mL.

In yet another embodiment according to the above aspects, the pharmaceutical formulation is dosed in an amount ranging from 0.5 mL to 12 mL (e.g., 0.5 mL to 1 ml, 1 mL to 1.5 ml, 1.5 mL to 2 ml, 2 mL to 2.5 ml, 2.5 mL to 3 ml, 3 mL to 3.5 ml, 3.5 mL to 4 ml, 4 mL to 4.5 ml, 4.5 mL to 5 ml, 5 mL to 5.5 ml, 5.5 mL to 6 ml, 6 mL to 6.5 ml, 6.5 mL to 7 ml, 7 mL to 7.5 ml, 7.5 mL to 8 ml, 8 mL to 8.5 ml, 8.5 mL to 9 ml, 9 mL to 9.5 ml, 9.5 mL to 10 ml, 10 mL to 10.5 ml, 10.5 mL to 11 ml, 11 mL to 11.5 ml, 11.5 mL to 12 ml, 1 ml to 11.5 mL, 1.5 ml to 11 ml, 2 ml to 10.5 ml, 2.5 ml to 10 ml, 3 ml to 9.5 ml, 3.5 ml to 9 ml, 4 ml to 8.5 ml, 4.5 ml to 8 ml, 5 ml to 7.5 ml, 5.5 ml to 7 ml, 6 ml to 7 ml, and all integers including and in between 0.5 mL to 12 mL). In some embodiments, the pharmaceutical formulation is dosed in an amount ranging from 0.5 mL to 1 mL. In some embodiments, the pharmaceutical formulation is dosed in an amount ranging from 1 mL to 1.5 ml. In some embodiments, the pharmaceutical formulation is dosed in an amount ranging from 1.5 mL to 2 ml. In some embodiments, the pharmaceutical formulation is dosed in an amount ranging from 2 mL to 2.5 ml. In some embodiments, the pharmaceutical formulation is dosed in an amount ranging from 2.5 mL to 3 ml. In some embodiments, the pharmaceutical formulation is dosed in an amount ranging from 3 mL to 3.5 ml. In some embodiments, the pharmaceutical formulation is dosed in an amount ranging from 3.5 mL to 4 ml. In some embodiments, the pharmaceutical formulation is dosed in an amount ranging from 4 mL to 4.5 ml. In some embodiments, the pharmaceutical formulation is dosed in an amount ranging from 4.5 mL to 5 ml. In some embodiments, the pharmaceutical formulation is dosed in an amount ranging from 5 mL to 5.5 ml. In some embodiments, the pharmaceutical formulation is dosed in an amount ranging from 5.5 mL to 6 ml. In some embodiments, the pharmaceutical formulation is dosed in an amount ranging from 6 mL to 6.5 ml. In some embodiments, the pharmaceutical formulation is dosed in an amount ranging from 6.5 mL to 7 ml. In some embodiments, the pharmaceutical formulation is dosed in an amount ranging from 7 mL to 7.5 ml. In some embodiments, the pharmaceutical formulation is dosed in an amount ranging from 7.5 mL to 8 ml. In some embodiments, the pharmaceutical formulation is dosed in an amount ranging from 8 mL to 8.5 ml. In some embodiments, the pharmaceutical formulation is dosed in an amount ranging from 8.5 mL to 9 ml. In some embodiments, the pharmaceutical formulation is dosed in an amount ranging from 9 mL to 9.5 ml. In some embodiments, the pharmaceutical formulation is dosed in an amount ranging from 9.5 mL to 10 ml. In some embodiments, the pharmaceutical formulation is dosed in an amount ranging from 10 mL to 10.5 ml. In some embodiments, the pharmaceutical formulation is dosed in an amount ranging from 10.5 mL to 11 ml. In some embodiments, the pharmaceutical formulation is dosed in an amount ranging from 11 mL to 11.5 ml. In some embodiments, the pharmaceutical formulation is dosed in an amount ranging from 11.5 mL to 12 ml. In some embodiments, the pharmaceutical formulation is dosed in an amount ranging from 1 ml to 11.5 mL. In some embodiments, the pharmaceutical formulation is dosed in an amount ranging from 1.5 ml to 11 ml. In some embodiments, the pharmaceutical formulation is dosed in an amount ranging from 2 ml to 10.5 ml. In some embodiments, the pharmaceutical formulation is dosed in an amount ranging from 2.5 ml to 10 ml. In some embodiments, the pharmaceutical formulation is dosed in an amount ranging from 3 ml to 9.5 ml. In some embodiments, the pharmaceutical formulation is dosed in an amount ranging from 3.5 ml to 9 ml. In some embodiments, the pharmaceutical formulation is dosed in an amount ranging from 4 ml to 8.5 ml. In some embodiments, the pharmaceutical formulation is dosed in an amount ranging from 4.5 ml to 8 ml. In some embodiments, the pharmaceutical formulation is dosed in an amount ranging from 5 ml to 7.5 ml. In some embodiments, the pharmaceutical formulation is dosed in an amount ranging from 5.5 ml to 7 ml. In some embodiments, the pharmaceutical formulation is dosed in an amount ranging from 6 ml to 7 ml. In some embodiments, the pharmaceutical formulation is dosed in an amount ranging from 2 mL to 8 mL.

In yet another embodiment according to the above aspects, the CNS disorder is selected from CDKL5 deficiency disorder (CDD), Angelman syndrome, Batten disease, Krabbe disease, Parkinson's disease, Alzheimer's disease, Spinal Muscular Atrophy (SMA) Types I, II, III, or IV, X-linked Myotubular Myopathy, Friedrich's Ataxia, Canavan's disease, Amyotrophic Lateral Sclerosis (ALS), Adrenoleukodystrophy, Huntington disease, Rett syndrome, or Spinocerebellar ataxia.

In yet another embodiment according to the above aspects, the rAAV comprises an AAV capsid and a vector genome packaged therein. In some embodiments, the AAV capsid is from an AAV of serotype 1, 2, 3, 4, 5, 6, 7, 8, 9, 10, 11, 12, rh10, hu37 (i.e., AAV1, AAV2, AAV3, AAV4, AAV5, AAV6, AAV7, AAV9, AAV10, AAV11, AAV12, AAVrh10, AAVhu37), or an engineered variant thereof. In an exemplary embodiment, the AAV capsid is an AAV serotype 9 (AAV9) capsid, an AAV9 variant capsid, an AAV serotype 8 (AAV8) capsid, an AAV8 variant capsid, or an AAV serotype hu37 (AAVhu37) capsid.

In yet another embodiment according to the above aspects, the CNS disorder is CDD and the rAAV comprises a nucleic acid sequence encoding a CDKL5 polypeptide. In yet another embodiment according to the above aspects, the CNS disorder is CDD and the rAAV is described in WO/2021/163322. In yet another embodiment according to the above aspects, the CNS disorder is CDD and the rAAV comprises an AAV9 capsid and a vector genome comprising SEQ ID NO: 1.

In another aspect, the present disclosure provides a process for preparing a pharmaceutical formulation for administration to the CNS of a subject suffering from a CNS disorder, wherein the pharmaceutical formulation comprises (i) a drug substance present at a minimum drug substance concentration, (ii) a maximum dose volume, and (iii) a maximum endotoxin concentration. In some embodiments, the process comprises: a) selecting a delivery route for the pharmaceutical formulation from intrathecal (IT), intracerebroventricular (ICV), and intra cisterna magna (ICM); b) based on the selected delivery route, selecting one or more excipients for the pharmaceutical formulation selected from a salt and/or a buffer; c) determining whether one or more of the excipients has a deleterious effect on one or more physical property of the pharmaceutical formulation selected from stability of the drug substance, viscosity, salt concentration, conductivity, trace metal content, osmolarity, density, pH, and biodistribution of the drug substance in the brain of a subject administered the pharmaceutical formulation; d) increasing the concentration of the one or more excipients in the pharmaceutical formulation to facilitate increasing drug substance concentration of the formulation if needed prior to purifying and/or concentrating the drug substance or spiking one or more of the excipients into the formulation after purifying and/or concentrating the drug substance; e) selecting low endotoxin raw materials for use in drug substance and pharmaceutical formulation manufacturing processes and evaluating strategies for further lowering raw materials endotoxins by cleaning and depyrogenation steps, wherein key raw materials endotoxin levels are <0.1 EU/mL for buffers, <0.01 EU/mL for pharmaceutical formulation vials, and/or <0.05 EU/mL for each piece of tubing or container that will contact the drug substance or the pharmaceutical formulation; and f) selecting low endotoxin raw materials for use in dose preparation and dose administration.

In some embodiments, the process disclosed herein results in extended stability of the pharmaceutical formulation. In some embodiments, the pharmaceutical formulation demonstrates stability for at least 2 months to at least 24 months (e.g., 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months). In some embodiments, the pharmaceutical formulation demonstrates stability for at least 12 months. In some embodiments, the pharmaceutical formulation demonstrates stability for at least 2 months to at least 24 months (e.g., 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months) in long term storage (e.g., ≤−60° C.). In some embodiments, the pharmaceutical formulation demonstrates stability for at least 12 months in long term storage at ≤−60° C.

In some embodiments, the drug substance is a recombinant adeno-associated vector (rAAV).

In some embodiments, the maximum dose volume is 12 mL.

In some embodiments, the maximum endotoxin concentration is 0.2 EU/mL.

In another aspect, the present disclosure provides a process for preparing a pharmaceutical formulation (comprising, e.g., a recombinant adeno-associated vector (rAAV)) for administration to the CNS of a subject.

In some embodiments, the pharmaceutical formulation may comprise (i) a drug substance present at a minimum drug substance concentration, (ii) a maximum dose volume, and (iii) a maximum endotoxin concentration.

In some embodiments, the process for preparing the pharmaceutical formulation comprises selecting a delivery route for the pharmaceutical formulation from intrathecal (IT), intracerebroventricular (ICV), and intra cisterna magna (ICM), then, based on the selected delivery route, selecting one or more excipients for the pharmaceutical formulation from a salt and/or a buffer. Having selected delivery route and identified appropriate excipients, the process may further entail determining whether one or more of the excipients has a deleterious effect on one or more physical property of the pharmaceutical formulation. Physical properties to be considered include stability of the drug substance, viscosity, salt concentration, conductivity, trace metal content, osmolarity, density, pH, and biodistribution of the drug substance in the brain of a subject administered the pharmaceutical formulation. The excipient(s) should be selected to have minimal deleterious effect(s). In addition, it should be determined whether the drug substance concentration in the pharmaceutical formulation can be increased by adding one or more of the excipients to the formulation prior to purifying and/or concentrating the drug substance or spiking one or more of the excipients into the formulation after purifying and/or concentrating the drug substance.

It is important to make sure that endotoxin risk is mitigated and controlled to low levels in both drug product and device components to enable dosing of pediatric populations and meet the USP <85> compendia.

In some embodiments, the process further comprises selecting low endotoxin raw materials for use in drug substance and pharmaceutical formulation manufacturing processes. In some embodiments, the process further comprises evaluating strategies for further lowering raw materials endotoxins by cleaning and depyrogenation steps, wherein key raw materials endotoxin levels are <0.1 EU/mL for buffers, <0.01 EU/mL for pharmaceutical formulation vials, and/or <0.05 EU/mL for each piece of tubing or container that will contact the drug substance or the pharmaceutical formulation. In some embodiments of the processes of the present disclosure, low endotoxin raw materials are selected for use in dose preparation.

In some embodiments, the process of the present disclosure further comprises selecting raw materials and components for use in dose preparation and administration, such that endotoxin concentration of the formulation is calculated according to Formula I:

$${e}_{D P} < \frac{0.2 \frac{E U}{kg} \cdot {m}_{b o d y} - {E}_{D e v}}{\frac{{D}_{b r a i n} * {m}_{B r a i n}}{{C}_{D P}}} , \qquad Formula I$$

wherein e_{DP} is the endotoxin concentration of the drug product; EU is endotoxin units; m_{body} is body weight of the subject; E_{Dev} is total endotoxin contribution from components used for dose preparation and dose administration of the formulation; m_{brain} is brain weight of the subject; D_{brain} is the dose to be delivered to CNS based on the m_{brain} and m_{body} of subject, e.g., as disclosed in Table 4; and C_{DP} is drug product concentration; and wherein 0.2 EU/kg is the tolerable endotoxin limit per body weight for Intrathecal Bolus Dose (<1 hr) (as per USP<85>). e_{DP} may be equivalent to E_{DP} (total endotoxin contribution of the drug product determined by adding the endotoxin contribution from the raw materials comprising the one or more excipients and the drug product) divided by V_{DP} (volume of the drug product).

In some embodiments of the process of the present disclosure, low endotoxin raw materials are alternatively or additionally implemented in dose administration to the subject to maximally reduce endotoxin exposure to the subject administered the pharmaceutical formulation.

In some embodiments of the process of the present disclosure, the drug substance is a rAAV, and the minimum C_{DP} is 1×10¹⁴ to 3×10¹⁴ GC/ml (e.g., 1×10¹⁴ to 1.2×10¹⁴ GC/ml, 1.2×10¹⁴ to 1.4×10¹⁴ GC/ml, 1.4×10¹⁴ to 1.6×10¹⁴ GC/ml, 1.6×10¹⁴ to 1.8×10¹⁴ GC/ml, 1.8×10¹⁴ to 2×10¹⁴ GC/ml, 2×10¹⁴ to 2.2×10¹⁴ GC/ml, 2.2×10¹⁴ to 2.4×10¹⁴ GC/ml, 2.4×10¹⁴ to 2.6×10¹⁴ GC/ml, 2.6×10¹⁴ to 2.8×10¹⁴ GC/ml, 2.8×10¹⁴ to 3×10¹⁴ GC/ml, 1.2×10¹⁴ to 3×10¹⁴ GC/ml, 1.4×10¹⁴ to 2.8×10¹⁴ GC/ml, 1.6×10¹⁴ to 2.6×10¹⁴ GC/ml, 1.8×10¹⁴ to 2.4×10¹⁴ GC/ml, and all integers including and in between 1×10¹⁴ to 2×10¹⁴ GC/ml). In some embodiments of the process of the present disclosure, the drug substance is a rAAV, and the minimum C_{DP} is 1×10¹⁴ to 1.2×10¹⁴ GC/ml. In some embodiments of the process of the present disclosure, the drug substance is a rAAV, and the minimum C_{DP} is 1.2×10¹⁴ to 1.4×10¹⁴ GC/ml. In some embodiments of the process of the present disclosure, the drug substance is a rAAV, and the minimum C_{DP} is 1.4×10¹⁴ to 1.6×10¹⁴ GC/ml. In some embodiments of the process of the present disclosure, the drug substance is a rAAV, and the minimum C_{DP} is 1.6×10¹⁴ to 1.8×10¹⁴ GC/ml. In some embodiments of the process of the present disclosure, the drug substance is a rAAV, and the minimum C_{DP} is 1.8×10¹⁴ to 2×10¹⁴ GC/ml. In some embodiments of the process of the present disclosure, the drug substance is a rAAV, and the minimum C_{DP} is 2×10¹⁴ to 2.2×10¹⁴ GC/ml. In some embodiments of the process of the present disclosure, the drug substance is a rAAV, and the minimum C_{DP} is 2.2×10¹⁴ to 2.4×10¹⁴ GC/ml. In some embodiments of the process of the present disclosure, the drug substance is a rAAV, and the minimum C_{DP} is 2.4×10¹⁴ to 2.6×10¹⁴ GC/ml. In some embodiments of the process of the present disclosure, the drug substance is a rAAV, and the minimum C_{DP} is 2.6×10¹⁴ to 2.8×10¹⁴ GC/ml. In some embodiments of the process of the present disclosure, the drug substance is a rAAV, and the minimum C_{DP} is 2.8×10¹⁴ to 3×10¹⁴ GC/ml. In some embodiments of the process of the present disclosure, the drug substance is a rAAV, and the minimum C_{DP} is 1.2×10¹⁴ to 3×10¹⁴ GC/ml. In some embodiments of the process of the present disclosure, the drug substance is a rAAV, and the minimum C_{DP} is 1.4×10¹⁴ to 2.8×10¹⁴ GC/ml. In some embodiments of the process of the present disclosure, the drug substance is a rAAV, and the minimum C_{DP} is 1.6×10¹⁴ to 2.6×10¹⁴ GC/ml. In some embodiments of the process of the present disclosure, the drug substance is a rAAV, and the minimum C_{DP} is 1.8×10¹⁴ to 2.4×10¹⁴ GC/ml. In some embodiments of the process of the present disclosure, the drug substance is a rAAV, and the minimum C_{DP} is at least 2×10¹⁴ GC/ml in the pharmaceutical formulation.

In some embodiments of the process of the present disclosure, the formulation comprises a total rAAV dose of 3.6×10¹⁴ to 3×10¹⁵ GC (e.g., 3.6×10¹⁴ to 4×10¹⁴ GC, 4×10¹⁴ to 5×10¹⁴ GC, 5×10¹⁴ to 6×10¹⁴ GC, 6×10¹⁴ to 7×10¹⁴ GC, 7×10¹⁴ to 8×10¹⁴ GC, 8×10¹⁴ to 9×10¹⁴ GC, 9×10¹⁴ to 1×10¹⁵ GC, 1×10¹⁵ to 2×10¹⁵ GC, 2×10¹⁵ to 3×10¹⁵ GC, 4×10¹⁵ to 2×10¹⁵ GC, 5×10¹⁴ to 1×10¹⁵ GC, 6×10¹⁴ to 9×10¹⁴ GC, and all integers including and in between 3.6×10¹⁴ to 3×10¹⁵ GC). In some embodiments of the process of the present disclosure, the formulation comprises a total rAAV dose of 3.6×10¹⁴ to 4×10¹⁴ GC. In some embodiments of the process of the present disclosure, the formulation comprises a total rAAV dose of 4×10¹⁴ to 5×10¹⁴ GC. In some embodiments of the process of the present disclosure, the formulation comprises a total rAAV dose of 5×10¹⁴ to 6×10¹⁴ GC. In some embodiments of the process of the present disclosure, the formulation comprises a total rAAV dose of 6×10¹⁴ to 7×10¹⁴ GC. In some embodiments of the process of the present disclosure, the formulation comprises a total rAAV dose of 7×10¹⁴ to 8×10¹⁴ GC. In some embodiments of the process of the present disclosure, the formulation comprises a total rAAV dose of 8×10¹⁴ to 9×10¹⁴ GC. In some embodiments of the process of the present disclosure, the formulation comprises a total rAAV dose of 9×10¹⁴ to 1×10¹⁵ GC. In some embodiments of the process of the present disclosure, the formulation comprises a total rAAV dose of 1×10¹⁵ to 2×10¹⁵ GC. In some embodiments of the process of the present disclosure, the formulation comprises a total rAAV dose of 2×10¹⁵ to 3×10¹⁵ GC. In some embodiments of the process of the present disclosure, the formulation comprises a total rAAV dose of 4×10¹⁵ to 2×10¹⁵ GC. In some embodiments of the process of the present disclosure, the formulation comprises a total rAAV dose of 5×10¹⁴ to 1×10¹⁵ GC. In some embodiments of the process of the present disclosure, the formulation comprises a total rAAV dose of 6×10¹⁴ to 9×10¹⁴ GC.

In some embodiments of the process of the present disclosure, the pharmaceutical formulation is dosed at 0.1×10¹² GC/g brain to 3×10¹² GC/g brain (e.g., 0.1×10¹² to 0.5×10¹² GC/g brain, 0.5×10¹² to 1×10¹² GC/g brain, 1×10¹² to 1.5×10¹² GC/g brain, 1.5×10¹² to 2×10¹² GC/g brain, 2×10¹² to 2.5×10¹² GC/g brain, 2.5×10¹² to 3×10¹² GC/g brain, 0.5×10¹² to 2.5×10¹² GC/g brain, 1×10¹² to 2×10¹² GC/g brain, and all integers including and in between 0.1×10¹² to 3×10¹² GC/g brain) of the subject. In some embodiments of the process of the present disclosure, the pharmaceutical formulation is dosed at 0.1×10¹² to 0.5×10¹² GC/g brain of the subject. In some embodiments of the process of the present disclosure, the pharmaceutical formulation is dosed at 0.5×10¹² to 1×10¹² GC/g brain of the subject. In some embodiments of the process of the present disclosure, the pharmaceutical formulation is dosed at 1×10¹² to 1.5×10¹² GC/g brain of the subject. In some embodiments of the process of the present disclosure, the pharmaceutical formulation is dosed at 1.5×10¹² to 2×10¹² GC/g brain of the subject. In some embodiments of the process of the present disclosure, the pharmaceutical formulation is dosed at 2×10¹² to 2.5×10¹² GC/g brain of the subject. In some embodiments of the process of the present disclosure, the pharmaceutical formulation is dosed at 2.5×10¹² to 3×10¹² GC/g brain of the subject. In some embodiments of the process of the present disclosure, the pharmaceutical formulation is dosed at 0.5×10¹² to 2.5×10¹² GC/g brain of the subject. In some embodiments of the process of the present disclosure, the pharmaceutical formulation is dosed at 1×10¹² to 2×10¹² GC/g brain of the subject. In some embodiments, the pharmaceutical formulation is dosed at 1×10¹² GC/g brain of the subject.

In some embodiments of the process of the present disclosure, the m_{brain} is 300 g-1400 g. In some embodiments of this process, the m_{brain} is 500 g-700 g (e.g., 500 g-550 g, 550 g-600 g, 600 g-650 g, 650 g-700 g, 550 g-650 g, 600 g-700 g, and all integers including and in between 500 g-700 g). In some embodiments of this process, the m_{brain} is 500 g-550 g. In some embodiments of this process, the m_{brain} is 550 g-600 g. In some embodiments of this process, the m_{brain} is 600 g-650 g. In some embodiments of this process, the m_{brain} is 650 g-700 g. In some embodiments of this process, the m_{brain} is 550 g-650 g. In some embodiments of this process, the m_{brain} is 600 g-700 g In some embodiments of the process of the present disclosure, the m_{body} is 2 kg to 25 kg (e.g., 2 kg to 5 kg, 5 kg to 7 kg, 7 kg to 9 kg, 9 kg to 12 kg, 12 kg to 15 kg, 15 kg to 20 kg, 20 kg to 25 kg, 5 kg to 20 kg, 7 kg to 15 kg, 9 kg to 15 kg, 12 kg to 20 kg, and all integers including and in between 2 kg to 25 kg). In some embodiments of the process of the present disclosure, the m_{body} is 2 kg to 5 kg. In some embodiments of the process of the present disclosure, the m_{body} is 5 kg to 7 kg. In some embodiments of the process of the present disclosure, the m_{body} is 7 kg to 9 kg. In some embodiments of the process of the present disclosure, the m_{body} is 9 kg to 12 kg. In some embodiments of the process of the present disclosure, the m_{body} is 12 kg to 15 kg. In some embodiments of the process of the present disclosure, the m_{body} is 15 kg to 20 kg. In some embodiments of the process of the present disclosure, the m_{body} is 20 kg to 25 kg. In some embodiments of the process of the present disclosure, the m_{body} is 5 kg to 20 kg. In some embodiments of the process of the present disclosure, the m_{body} is 7 kg to 15 kg. In some embodiments of the process of the present disclosure, the m_{body} is 9 kg to 15 k. In some embodiments of the process of the present disclosure, the m_{body} is 12 kg to 20 kg. In some embodiments of the process of the present disclosure, the m_{body} is 3 kg to 6 kg (e.g., 3 kg to 3.5 kg, 3.5 kg to 4 kg, 4 kg to 4.5 kg, 4.5 kg to 5 kg, 5.5 kg to 6 kg, 3.5 kg to 5.5 kg, 4 kg to 5 kg, 3.5 kg to 5.5 kg, 4.5 kg to 5.5 kg, and all integers including and in between 3 kg to 6 kg). In some embodiments of the process of the present disclosure, the m_{body} is 3 kg to 3.5 kg. In some embodiments of the process of the present disclosure, the m_{body} is 3.5 kg to 4 kg. In some embodiments of the process of the present disclosure, the m_{body} is 4 kg to 4.5 kg. In some embodiments of the process of the present disclosure, the m_{body} is 4.5 kg to 5 kg. In some embodiments of the process of the present disclosure, the m_{body} is 5.5 kg to 6 kg. In some embodiments of the process of the present disclosure, the m_{body} is 3.5 kg to 5.5 kg. In some embodiments of the process of the present disclosure, the m_{body} is 4 kg to 5 kg. In some embodiments of the process of the present disclosure, the m_{body} is 3.5 kg to 5.5 kg. In some embodiments of the process of the present disclosure, the m_{body} is 4.5 kg to 5.5 kg.

In some embodiments of the process of the present disclosure, the V_{DP} is 1.8 to 12 ml (e.g., 1.8 to 2.4 ml, 2.4 to 4.8 ml, 4.8 to 7.5 ml, 7.5 to 10 ml, 10 to 12 ml, 2.4 to 10 ml, 4.8 ml to 10 ml, 2.4 ml to 7.5 ml, 7.5 ml to 12 ml, and all values to the one-tenth decimal value including and in between 1.8 ml to 12 ml). In some embodiments of the process of the present disclosure, the V_{DP} is 1.8 to 2.4 ml. In some embodiments of the process of the present disclosure, the V_{DP} is 2.4 to 4.8 ml. In some embodiments of the process of the present disclosure, the V_{DP} is 4.8 to 7.5 ml. In some embodiments of the process of the present disclosure, the V_{DP} is 7.5 to 10 ml. In some embodiments of the process of the present disclosure, the V_{DP} is 10 to 12 ml. In some embodiments of the process of the present disclosure, the V_{DP} is 2.4 to 10 ml. In some embodiments of the process of the present disclosure, the V_{DP} is 4.8 ml to 10 ml. In some embodiments of the process of the present disclosure, the V_{DP} is 2.4 ml to 7.5 ml. In some embodiments of the process of the present disclosure, the V_{DP} is 7.5 ml to 12 ml. In some embodiments of the process of the present disclosure, the V_{DP} is 10 ml.

In some embodiments of the process of the present disclosure, the maximum endotoxin concentration of the pharmaceutical formulation is ≤0.2 EU/ml. In some embodiments of the process of the present disclosure, the maximum endotoxin concentration of the pharmaceutical formulation is ≤0.1 EU/ml.

In some embodiments of the process of the present disclosure, the e_{DP} endotoxin limit is 0.08 to 0.74 EU/ml (e.g., 0.08 to 0.15 EU/ml, 0.15 to 0.3 EU/ml, 0.3 to 0.5 EU/ml, 0.5 to 0.74 EU/ml, 0.15 to 0.5 EU/ml, 0.3 to 0.74 EU/ml, 0.15 to 0.5 EU/ml, and all values to the one-hundredth decimal value including and in between 0.08 to 0.74 EU/ml). In some embodiments of the process of the present disclosure, the e_{DP} endotoxin limit is 0.08 to 0.15 EU/ml. In some embodiments of the process of the present disclosure, the e_{DP} endotoxin limit is 0.15 to 0.3 EU/ml. In some embodiments of the process of the present disclosure, the e_{DP} endotoxin limit is 0.3 to 0.5 EU/ml. In some embodiments of the process of the present disclosure, the e_{DP} endotoxin limit is 0.5 to 0.74 EU/ml. In some embodiments of the process of the present disclosure, the e_{DP} endotoxin limit is 0.15 to 0.5 EU/ml. In some embodiments of the process of the present disclosure, the e_{DP} endotoxin limit is 0.3 to 0.74 EU/ml. In some embodiments of the process of the present disclosure, the e_{DP} endotoxin limit is 0.15 to 0.5 EU/ml.

In some embodiments of the process of the present disclosure, the E_{dev} is up to 0.32 EU. In some embodiments, the process of the present disclosure further comprises lowering the E_{dev} by cleaning and depyrogenating the components used for dose preparation and dose administration of the formulation.

In some embodiments of the process of the present disclosure, the E_{dev} comprises <0.01 EU/mL for pharmaceutical formulation vials, and/or <0.05 EU/mL for each piece of tubing or container that will contact the drug substance or the pharmaceutical formulation.

In some embodiments of the process of the present disclosure, the E_{dev} comprises <0.1 EU/mL for at least one buffer used as raw material.

In some embodiments of the process of the present disclosure, the rAAV comprises an AAV capsid and a vector genome packaged therein. In some embodiments, the AAV capsid is from an AAV of serotype 1, 2, 3, 4, 5, 6, 7, 8, 9, 10, 11, 12, rh10, hu37 (i.e., AAV1, AAV2, AAV3, AAV4, AAV5, AAV6, AAV7, AAV9, AAV10, AAV11, AAV12, AAVrh10, AAVhu37), or an engineered variant thereof.

In some embodiments of the process of the present disclosure, the CNS disorder is CDD and the rAAV comprises a nucleic acid sequence encoding a CDKL5 polypeptide. In some embodiments of the process of the present disclosure, the CNS disorder is CDD and the rAAV is described in WO/2021/163322. In some embodiments of the process of the present disclosure, the CNS disorder is CDD and the rAAV comprises an AAV9 capsid and a vector genome comprising SEQ ID NO: 1. These and other aspects and features of the invention are described in the following sections of the present disclosure.

### BRIEF DESCRIPTION OF THE DRAWINGS

FIG. 1 is a graph showing diffusion coefficient analysis of rAAV concentration in presence of salt only (100 mM NaCl) or salt with additive (130 mM NaCl and 5% sorbitol). The diffusion coefficient is indicated on the y-axis and the concentration of rAAV in terms of genomic titer is indicated on the x-axis.

FIGS. 2A-2D are histogram plots evaluating genome titer at 37° C. (FIG. 2A), in vitro mRNA expression (FIG. 2B; F2 was not tested for in vitro mRNA expression for the 10× freeze-thaw cycle, as indicated), capsid impurity level when subjected to capillary electrophoresis (CE)-SDS (FIG. 2C), and percentage of high molecular weight aggregation determined using size-exclusion chromatography (SEC) (FIG. 2D) of three formulations in varying conditions (baseline timepoint denoted as “0” or “T0”, 1 week at 37° C. (denoted as “37C 1WK”), 2 weeks at 37° C. (denoted as “37C 2WK”), 8 weeks at 4° C. (denoted as “4C 8WK”), 8 weeks at 25° C. (denoted as “25C 8WK”), or after 10 freeze and thaw cycles (denoted as “10× F/T” or “F/T 10×”), as indicated on the x-axis. The three tested formulations were F1 (10 mM sodium phosphate, 135 mM NaCl, 0.27M Sorbitol, 0.001% v/v poloxamer 188, pH 7.2); F2 (10 mM sodium phosphate, 135 mM NaCl, 0.54M Sorbitol, 0.001% v/v poloxamer 188, pH 7.2); and F3 (10 mM Tris, 130 mM NaCl, 2 mM KCl, 1 mM MgCl₂, 2 mM CaCl₂, 0.27M Sorbitol, 0.001% v/v poloxamer 188, pH 6.8).

FIGS. 3A-3D are fluorescence microscopy images showing effect on CDKL5 mRNA expression in brain tissue of animals injected with the rAAV-CDKL5 in the three different formulations disclosed herein as follows: F1: 10 mM sodium phosphate, 135 mM NaCl, 0.27M Sorbitol, 0.001% v/v poloxamer 188, pH 7.2 (FIG. 3B); F2: 10 mM sodium phosphate, 135 mM NaCl, 0.54M Sorbitol, 0.001% v/v poloxamer 188, pH 7.2 (FIG. 3C); and F3: 10 mM Tris, 130 mM NaCl, 2 mM KCl, 1 mM MgCl₂, 2 mM CaCl₂), 0.27M Sorbitol, 0.001% v/v poloxamer 188, pH 6.8 (FIG. 3A), and F1 with hydroxychloroquine at 25 mg/kg (FIG. 3D). The expression of CDKL5 in brain tissue is indicated by arrows.

FIG. 4 is a chart showing the outline of drug substance manufacturing and drug product manufacturing enabling tight control of AAV concentration. Abbreviation used in FIG. 4: formulation tangential flow filtration (FTFF); tangential flow filtration (TFF); at status quantitative PCR (STAT qPCR); drug substance (DS); drug product (DP); droplet digital PCR (ddPCR).

FIG. 5 is a chart showing estimated dose volume of drug product per minimum patient's body weight (ml/kg) as indicated on y-axis corresponding to patient age as indicated on x-axis. The low and high brain weight groups are as indicated.

FIG. 6 is a graph showing the contribution of drug product (DP) endotoxin level to the endotoxin risk of patients administered drug dosage based on body weight. The assumed drug product concentration is 1×10¹⁴ GC/ml, and the device endotoxin contribution is 0.5 endotoxin unit (EU)/device. The total endotoxin deliverable to patients corresponding to body weight, based on the USP<85> EU limit of 0.2 EU/Kg is indicated as solid black line. The total endotoxin (EU) delivered to the patients indicated on y-axis, corresponding to patient body weight (kg) as indicated on x-axis, based on concentration of endotoxin in the drug product (0.025 EU/ml, 0.05 EU/ml, 0.1 EU/ml and 0.2 EU/ml), are as shown.

FIG. 7 is a graph showing the contribution of endotoxin from device to the endotoxin risk of patients administered drug dosage based on body weight. The assumed drug product concentration is 1×10¹⁴ GC/ml, and the drug product endotoxin contribution is 0.1 EU/ml. The total endotoxin deliverable to patients corresponding to body weight, based on the EU limit of 0.2 EU/Kg is indicated as solid black line. The total endotoxin (EU) delivered to the patients as indicated on y-axis, corresponding to patient body weight (kg) as indicated on the x-axis, based on concentration of endotoxin in the drug product (0.25 EU/device, 0.5 EU/device, 1.0 EU/device and 2.5 EU/device), are shown.

FIG. 8 is a graph showing the contribution of endotoxin from device to the endotoxin risk of patients administered drug dosage based on body weight. The assumed high dose of the drug product is 1.2×10¹² GC/g brain weight, drug product concentration of 1×10¹⁴ GC/ml, measured endotoxin concentration of the drug product (eDP) is <0.1 EU/ml and the endotoxin contribution from the device is <0.2601 EU/device. The total endotoxin deliverable to patients corresponding to body weight, based on the USP<85> EU limit of 0.2 EU/Kg is indicated as solid black line. The total endotoxin (EU) delivered to the patients as indicated on y-axis, corresponding to patient body weight (kg) as indicated on the x-axis, for low brain weight and high brain weight categories, is shown.

### DETAILED DESCRIPTION OF THE DISCLOSURE

For efficacious gene therapy for certain rare disorders, there can be several pre-clinical and clinical challenges. One such disorder is CDKL5 deficiency disorder (CDD), a rare neurodevelopmental disease caused by mutations in the CDKL5 gene which can manifest in a broad range of clinical symptoms and severity. Hallmarks of CDD include infantile-onset refractory epilepsy, developmental delay, intellectual disability, visual impairment, lack of speech, hypotonia, motor dysfunction, sleep disturbances, gastrointestinal dysfunction, and breathing difficulties. Although rare, the occurrence is believed to be approximately 1 in 40,000-60,000 live births, making it one of the most common forms of genetic epilepsy. AAV for the treatment of CDD and formulations comprising the same are described in WO/2021/163322 and WO/2023/023590, respectively, the disclosures of which are herein incorporated by reference in their entireties.

For effective treatment of CDD, the applicant has determined it is important to deliver the rAAV directly to the brain, e.g., through intracisternal magna (ICM) administration, to achieve widespread distribution and neuron transduction. The applicant has also determined it is important to use a high dose of rAAV, e.g., at least about 1×10¹² genome copies (GC)/gram of brain tissue, which can be dose dependent based upon patient brain mass. In addition, since the cerebrospinal fluid (CSF) volume is relatively low, a low dose volume (<12 mL) is desirable to prevent disruption of the pressure balance in the central nervous system (CNS) region. And because a high dose of rAAV is desired despite a low dose volume, the applicant has discovered it is necessary to create a formulation comprising a very high rAAV concentration. Finally, it is critical to utilize a drug product formulation that is safe for administration to the CNS and compatible with CSF.

Beyond the aforementioned obstacles, the difficulty associated with developing a liquid formulation suitable for treating CNS disorders is compounded by the fact that only a very low level of endotoxin (<0.2 EU/kg body weight/h) is allowed for intrathecal administration. See United States Pharmacopeia (USP), Chapter <85>, Bacterial Endotoxins Test, 161-167, U.S. Pharmacopeial Convention, Rockville, MD, 2015. See also European Pharmacopoeia 5.0, Chapter 2.6.14 (Eur.Ph.2.6.14). This is far lower than is allowed for any non-intrathecal parenteral route of administration (<5.0 EU/kg body weight/h). Moreover, in pediatric patients, dosing requires very tight control over drug product concentration, dose volume, and endotoxin levels, amongst other impurities. Finally, pediatric patients have the lowest body weight of any age group and thus extremely low endotoxin levels in the drug product to meet the requirements of USP, Chapter <85> and Eur.Ph.2.6.14.

In view of the above, Applicant has designed formulations comprising high concentrations of recombinant AAV and low levels of endotoxin. These formulations can be successfully administered (e.g., intrathecally) to the subjects for the treatment of rare genetic disorders of the CNS.

The present disclosure provides methods for preparing pharmaceutical formulations comprising rAAV suitable for administration to the CNS via intrathecal (IT), intracerebroventricular (ICV), and/or intracisternal magna (ICM) routes. Also described herein are pharmaceutical formulations comprising rAAV suitable for administration to the CNS and methods of their use in the treatment of CNS disorders, e.g., rare genetic CNS disorders such as CDKL5 deficiency disorder (CDD).

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Adeno-associated virus (AAV): A small, replication-defective, non-enveloped virus that infects humans and some other primate species. AAV is not known to cause disease and elicits a very mild immune response. Gene therapy vectors that utilize AAV can infect both dividing and quiescent cells and can persist in an extrachromosomal state without integrating into the genome of the host cell. These features make AAV an attractive viral vector for gene therapy. There are currently 13 recognized serotypes of AAV (AAV1-13). The term “AAV particle” refers to an AAV capsid and a genome packaged therein.

Administration/Administer: To provide or give a subject an agent, such as a therapeutic agent (e.g., a recombinant AAV), by any effective route. Exemplary routes of administration include, but are not limited to, injection (such as subcutaneous, intramuscular, intradermal (ID), intraperitoneal (IP), intrathecal (IT), intracerebroventricular (ICV), intracisternal magna (ICM), or intravenous (IV) administration), oral, intraductal, sublingual, rectal, transdermal, intranasal, vaginal and inhalation routes.

Central nervous system (CNS) generally refers to the spinal cord and brain and contrasts with the “peripheral nervous system” which excludes the spinal cord and brain. There are different cell types within the central nervous system, including neuronal cells and glial cells. The glia in mature systems include astrocytes, oligodendrocytes, and microglial cells. The AAV capsids for the vectors used in the present invention are preferably selected from among those which will transduce and/or express in at least one of these cell types of the central nervous system.

Endotoxin generally refers to toxic substance(s) composed of lipopolysaccharides (LPS), which comprises a lipid portion (lipid A), a core polysaccharide, and an O-specific polysaccharide chain. Endotoxins are released when a bacterial cell wall is disrupted, such as during bacterial lysis, growth, or death. Because endotoxins may stimulate the innate immune system, their removal from medical products, especially injectable drugs and medical devices, is important. Methods for determining the amount of endotoxin are known in the art, e.g., one may use a limulus amoebocyte lysate (LAL) test. For instance, the amount of endotoxin is measured using a traditional LAL kinetic chromogenic assay, e.g., such as the highly-sensitive Endochrome-K™ assay available from Charles River Laboratories International, Wilmington, MA.

Intrathecal delivery/Intrathecal administration generally refers to a route of administration for drugs via an injection, e.g., an infusion, into the spinal canal, more specifically into the subarachnoid space so that it reaches the cerebrospinal fluid (CSF). Intrathecal delivery may include lumbar puncture, intraventricular (including intracerebroventricular (ICV)), suboccipital/intracisternal, and/or CI-2 puncture. For example, material (e.g., a pharmaceutical formulation) may be introduced for diffusion throughout the subarachnoid space by means of lumbar puncture. In another example, injection may be into the cisterna magna.

Intracisternal magna delivery/Intracisternal magna administration/injection into the cisterna magna generally refers to a route of administration for drugs directly into the cerebrospinal fluid of the cisterna magna cerebellomedularis, more specifically via a suboccipital puncture or by direct injection into the cisterna magna or via permanently positioned tube.

Preventing, treating or ameliorating a disease: “Preventing” a disease (such as CDD) refers to inhibiting the full development of a disease. “Treating” refers to a therapeutic intervention that ameliorates a sign or symptom of a disease or pathological condition (such as CDD) after it has begun to develop. “Ameliorating” refers to the reduction in the number or severity of signs or symptoms of a disease (such as CDD).

Recombinant: A recombinant nucleic acid molecule is one that has a sequence that is not naturally occurring or has a sequence that is made by an artificial combination of two otherwise separated segments of sequence. This artificial combination can be accomplished by chemical synthesis or by the artificial manipulation of isolated segments of nucleic acid molecules, such as by genetic engineering techniques.

Similarly, a recombinant virus is a virus comprising sequence (such as genomic sequence) that is non-naturally occurring or made by artificial combination of at least two sequences of different origin. The term “recombinant” also includes nucleic acids, proteins and viruses that have been altered solely by addition, substitution, or deletion of a portion of a natural nucleic acid molecule, protein or virus. As used herein, “recombinant AAV” (also rAAV, used interchangeably with rAAV particle) refers to an AAV particle in which a recombinant nucleic acid molecule (e.g., a recombinant nucleic acid molecule encoding CDKL5) has been packaged.

Serotype: A group of closely related microorganisms (such as viruses) distinguished by a characteristic set of antigens.

Subject: Living multi-cellular vertebrate organisms, a category that includes human and non-human mammals. In some embodiments, the subject is a human. In one embodiment, the human subject is an adult subject, i.e., a human subject greater than 18 years old. In one embodiment, the human subject is a pediatric subject, i.e., a human subject of ages 0-18 years old inclusive.

Therapeutically effective amount: A quantity of a specified pharmaceutical or therapeutic agent (e.g., a recombinant AAV) sufficient to achieve a desired effect in a subject, or in a cell, being treated with the agent. The effective amount of the agent will be dependent on several factors, including, but not limited to the subject or cells being treated, and the manner of administration of the therapeutic composition.

Excipient: Ingredients other than the active pharmaceutical ingredient (API) or drug substance present in a finished pharmaceutical drug formulation. The excipient can be one or more of lubricant, diluent, binder, flavoring, buffer, coating and coloring agent for the finished pharmaceutical drug formulation. In some embodiments of the various aspects of the present disclosure, the one or more excipients of the formulation are selected based on the route of administration of the finished pharmaceutical drug formulation to the subject, the concentration of the drug product in the finished pharmaceutical drug formulation, the physical properties of the drug product (e.g. capsid serotype of a rAAV drug product) within the finished pharmaceutical drug formulation, or a combination thereof.

In some embodiments of the various aspects of the present disclosure, endotoxin contribution from an excipient in the pharmaceutical drug formulation is the total endotoxin contribution from the raw materials/components used for making or preparing the one or more excipients, i.e., the level of total endotoxin present in the amount of the various components used to make the excipient. For example, the endotoxin contribution from a buffer (an excipient) comprising 10 mM sodium phosphate, 135 mM NaCl, 0.27M Sorbitol, 0.001% v/v poloxamer 188, is the total endotoxin amount calculated by adding up the endotoxin levels in the respective amounts of each buffer component i.e., sodium phosphate, NaCl, Sorbitol, poloxamer 188, that were combined to form the buffer.

Drug product (DP) is a finished pharmaceutical drug formulation comprising a drug substance or API and one or more excipients. A drug product may be liquid or solid, lyophilized or reconstituted in liquid, e.g., for administration. Drug product or DP is used interchangeably with finished pharmaceutical drug formulation.

In some embodiments of the various aspects of the present disclosure, the endotoxin contribution of the drug product is the total endotoxin calculated by adding the total endotoxin from the formulation comprising the drug substance or API, in some cases a rAAV drug substance, and the one or more excipients.

Device components refer to machines or apparatuses and parts thereof, for preparing and administering (e.g., injecting) a drug substance within the drug product. In some embodiments of the present disclosure, one or more endotoxins can be introduced into the drug product through one or more device components that physically contacts the one or more excipients, the drug substance, or the drug product, during preparation or administration of the drug product.

In some embodiments of the various aspects of the present disclosure, the endotoxin contribution from device components is calculated by adding up the endotoxin levels of each machine or apparatus or part thereof that physically contacts the one or more excipients, the drug substance, or the drug product.

Brain mass (m_{brain}): weight of the brain of a subject measured by methods used in the art including but not limited to magnetic resonance imaging (MRI) and computed tomography (CT) scans.

Body mass (m_{body}): weight of the physical body of the subject measured by standing on a suitable digital or mechanical weighing scale. In some embodiments, the physical body of the subject is weighed without clothing.

Drug product concentration (C_{DP}) is the number of genome copies of rAAV particles present per milliliter of the drug formulation.

Dose to be delivered to CNS (D_{brain}), in reference to rAAV drug substance or drug product, is the total number of genome copies of rAAV calculated by multiplying the C_{DP} by the volume of the drug product particles that is delivered or injected into the brain of a subject.

Vector: A vector is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An expression vector is a vector that contains the necessary regulatory sequences to allow transcription and translation of inserted gene or genes. In some embodiments herein, the vector is an AAV vector. The term “vector” may also be used in the general sense of an agent that carries genetic information or material, for example, to introduce the genetic material to a cell or organism. Thus, the term “vector” may refer to, e.g., a rAAV particle (as in the case of a “viral vector”), or to a nucleic acid molecule encoding one or more proteins of a rAAV particle (such as a plasmid), or to a nucleic acid molecule encoding a gene to be carried by a “viral vector” (i.e., a “vector genome”). Persons skilled in the art will readily appreciate the generic uses of the term “vector” as an agent that carries genetic information or material as well as the more specific uses of the term “vector” referring to a plasmid or a vector genome within a rAAV particle.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

In the present disclosure, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within the present disclosure, embodiments have been described and depicted in a way that enables a clear and concise disclosure to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present disclosure also includes the specific quantitative value itself, unless specifically stated otherwise. As used herein, the term “about” refers to a ±10% variation from the nominal value unless otherwise indicated or inferred.

Unless otherwise noted, where the term “between” is used to refer to a numerical range, the range includes the specified endpoints. For example, the range “between 1×10¹⁴ to 3×10¹⁵ GC/ml” includes 1×10¹⁴, 3×10¹⁵, and values greater than 1×10¹⁴ but less than 3×10¹⁵.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including” is intended merely to illustrate better the present invention and does not pose a limitation on the scope of the invention unless claimed. No language in the specification should be construed as indicating any non-claimed element as essential to the practice of the present invention.

### EXAMPLES

The disclosure now being generally described, will be more readily understood by reference to the following examples, which are included merely for purposes of illustration of certain aspects and embodiments of the present disclosure, and are not intended to limit the scope of the disclosure in any way.

### Example 1

The purpose of this example is to demonstrate how an rAAV-comprising pharmaceutical formulation compatible for an intracisternal magna (ICM) delivery was developed.

### Concentration Study:

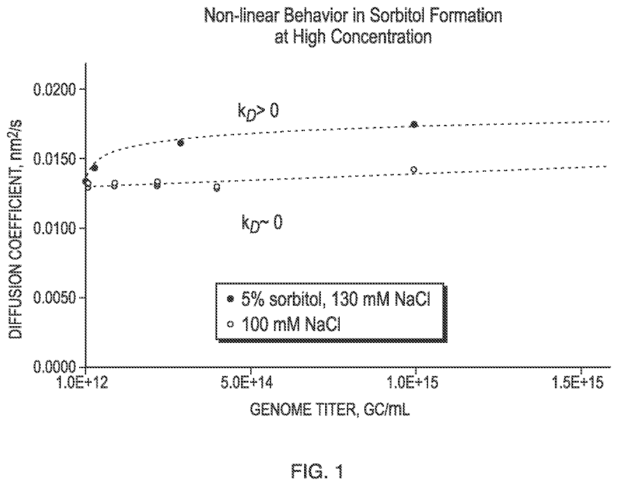

Recombinant AAV are normally formulated with excipients such as salt and buffer. To evaluate the impact of various excipient components on formulation tangential flow filtration (FTFF) concentration, excipients used in clinical trials leveraging intrathecal (IT), intracerebroventricular (ICV), and intracisternal magna (ICM) delivery were identified and examined. A diffusion coefficient analysis showed that selected excipients severely limited the FTFF concentration process (FIG. 1), as evident from the non-linear behavior in sorbitol containing formulation at high rAAV concentration of 5×10¹⁴ GC/ml and above. The increase in diffusion coefficient showed a linear relationship to the increasing genome titer, in formulations without the excipient, sorbitol. Removal of the selected excipients during the FTFF concentration enabled the achievement of concentrations greater than 5×10¹⁴ GC/mL. Thus, to allow for high concentrations of rAAV in the pharmaceutical formulation, the excipient spike step should be performed after the concentration step.

### Formulation Stability:

As a next step, three formulations (F1, F2, and F3) were evaluated for various properties indicative of stability, including genome titer, in vitro mRNA expression, integrity when subjected to capillary electrophoresis (CE)-SDS, and percent aggregation as determined by size exclusion chromatography. The components of the F1, F2, and F3 formulations were as follows:

F1: 10 mM sodium phosphate, 135 mM NaCl, 0.27M Sorbitol, 0.001% v/v poloxamer 188, pH 7.2.

F2: 10 mM sodium phosphate, 135 mM NaCl, 0.54M Sorbitol, 0.001% v/v poloxamer 188, pH 7.2.

F3: 10 mM Tris, 130 mM NaCl, 2 mM KCl, 1 mM MgCl₂, 2 mM CaCl₂), 0.27M Sorbitol, 0.001% v/v poloxamer 188, pH 6.8.

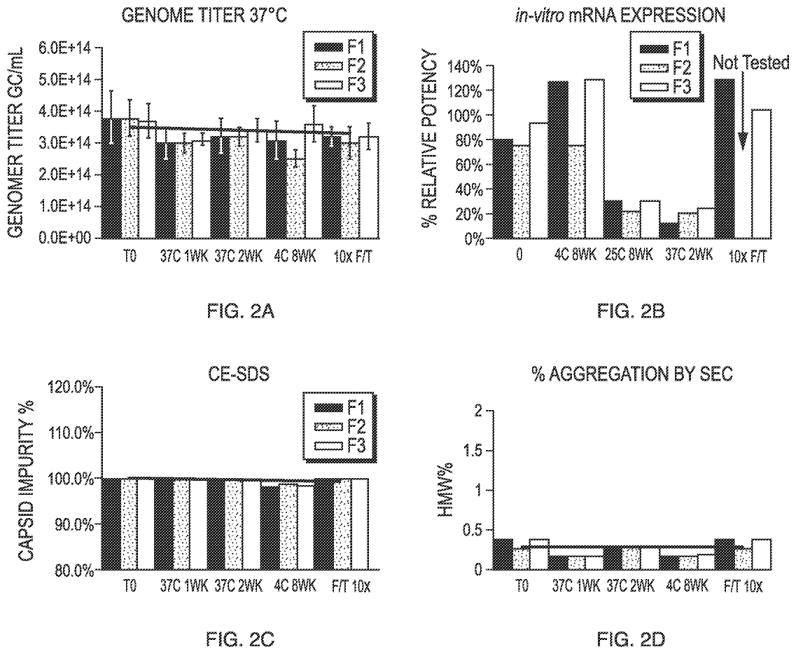

As shown in FIG. 2A-2D, all three formulations exhibited similar stability. At 25° C. and 37° C., there was a decline in mRNA potency after 8 weeks and 2 weeks, respectively, regardless of formulation. At 4° C., there was stability out to 8 weeks with all three formulations. Up to 10 freeze/thaw cycles were well tolerated and long term-stability (12 months) at −60° C. was observed. As indicated in FIG. 2B, the formulation 2 (F2) was not tested for in vitro mRNA expression for the 10× freeze-thaw cycle.

### Evaluation for Brain Distribution in Mice Following ICM Delivery:

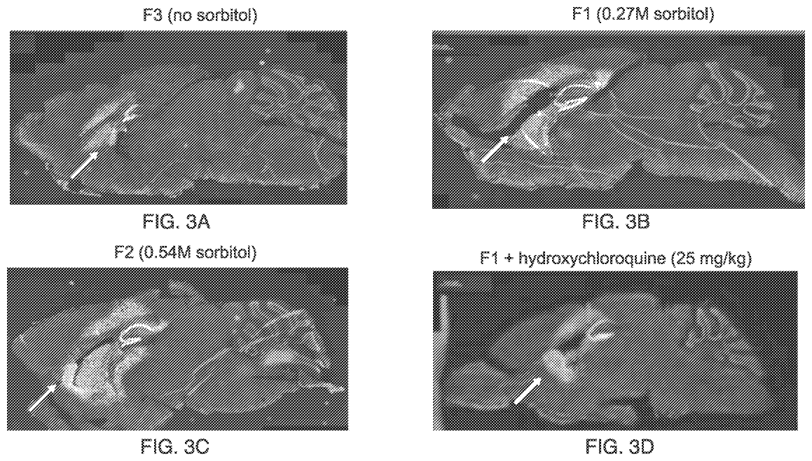

Next, two excipients were evaluated in the context of CDKL5-specific mRNA expression in mice brains, as shown in FIG. 3A-3D. In this experiment, excipients 1 and 2 were sorbitol and hydroxychloroquine, respectively. As indicated in FIGS. 3A-3D, CDKL5 specific mRNA expression was seen in animal brains administered with all formulations of the present disclosure as compared to control untreated animals (data not disclosed). Further, the CDKL5 expression was higher in brain of animals treated with formulations, F1 and F2 comprising 0.27M and 0.54M sorbitol, respectively, (FIGS. 3B-3C), as compared to formulation without sorbitol (F3, FIG. 3A), showing a positive effect of sorbitol in preserving the drug product stability and integrity. Combining a second excipient, hydroxychloroquine with formulation 1 containing sorbitol (FIG. 3D) did not show better CDKL5 mRNA expression, as compared to formulations containing sorbitol alone.

### AAV Concentration Control Strategy:

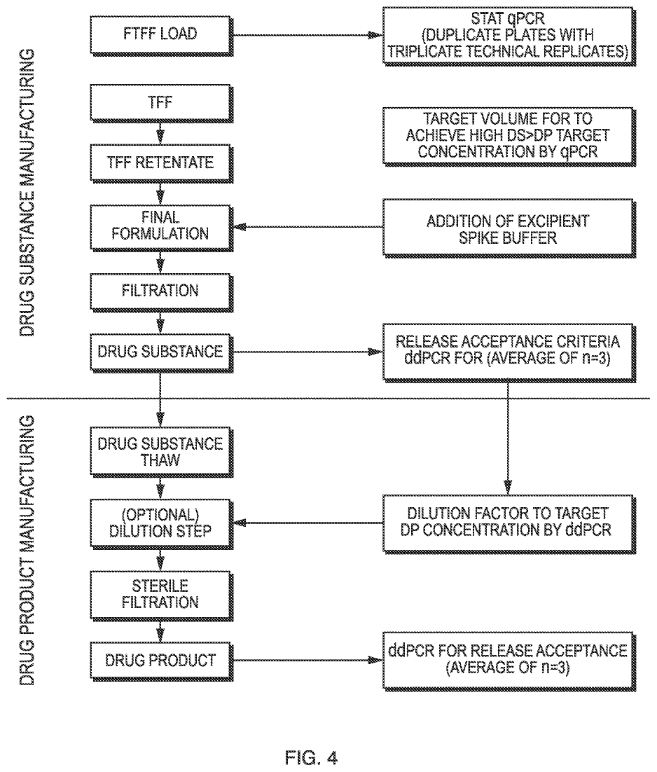

High in-process qPCR variability led to the establishment of a unique strategy to robustly target drug substance and drug product concentrations exceeding 1×10¹⁴ GC/mL utilizing replicate measures to reduce measurement variability impact and enable tight targeting of drug product concentration. Given the high variability with qPCR, digital PCR (dPCR) is preferred for in-process measures, as it shows reduced variability and improved accuracy and precision, similar to ddPCR. An outline of drug substance manufacturing and drug product manufacturing enabling tight control of AAV concentration is shown in FIG. 4.

### Strategies to Reduce Endotoxin Levels:

As required by Chapter <85> of the United States Pharmacopeia (USP), the level of acceptable endotoxin for intrathecal delivery is <0.2 EU/kg body weight/hr.

To ensure low endotoxin levels in a pharmaceutical formulation (e.g., drug product), the instant inventors conducted a thorough evaluation of drug substance and drug product manufacturing process components to identify which ones can contribute to unwanted endotoxin accumulation. By virtue of this evaluation, the inventors discovered the importance of selecting low endotoxin raw materials. As a general rule, for the selection of raw materials for product development, a buffer acceptance criteria of <0.1 EU/mL was used. For DP vials, a general acceptance criteria of <0.01 EU/mL was established. Finally, with respect to stoppers, it was determined that, generally, an acceptable level is <0.05 EU/mL. Assessment of materials used upstream of drug product manufacturing also should be conducted. Beyond the importance of raw materials selection, the inventors assessed cleaning and wash steps to improve endotoxin clearance; depyrogenation is an important tool to further reduce endotoxin levels as needed.

For component screening, a low limit of quantitation (LOQ) and sensitive endotoxin method was developed which leveraged a LAL-based kinetic chromogenic method (Endochrome-K™ assay available from Charles River Laboratories International, Wilmington, MA). The method is highly sensitive (detection of 0.005 EU/mL). Here, the inventors used a very low dilution factor to enable a very low LOQ of <0.01 EU/mL. It was determined that minimal test volumes and a low dilution was critical to enabling a low LOQ.

The final components were screened and selected via the aforementioned, highly-sensitive endotoxin method following USP <161> to achieve <0.5 EU/device setup. Following screening, three different combinations were identified which met acceptance criteria:

Combination 1: SmartSite Universal Vial Access (Carefusion, MV0400), Texium Closed Male Luer (BD, 10012241-0500), BD Quincke Spinal Needle (BD, 405074), Millex-GP 0.2 μm filter (Millipore, 5LGPM33RS), Microbore extension set 6.5 in (17 cm) (Baxter, 2N3339), Female-luer to female-luer connector (Fisher Scientific, NC1652395), and 10-mL syringe (BD 309642).

Combination 2: Sterile Vial Adapter 20 (West Pharmaceuticals, 8072036), Texium CSTD (BD, 10012241-0500), BD Quincke Spinal Needle (BD, 405074), Small-bore extension set 5 in (13 cm) (BBraun, 471954), and HSW Softject 10-mL (HSW, RL10).

Combination 3: Equashield Vial Adapter (Equashield, VA-20/2), Female LL Connector (Equashield, FC-1), Reli spinal needle (Reli, SN25G351), BBraun small bore extension tubing (BBraun, 471954), 0.2 μm filter (BBraun PFE2000), Normject syringe (Normject, 4100-X00V0).

Closed-system transfer devices (CSTD) showed higher endotoxin levels than other administration components. The number of components used and contact duration should thus be evaluated in the dose preparation and administration procedure.

### Optimizing Dose Volume, Concentration, and Endotoxin Levels for Pediatric Patients:

Significant challenges are faced with the pediatric patients who have the highest ratio of brain weight to body weight. A low drug product concentration requires a higher dose volume which can disrupt the CSF pressure balance. Meanwhile, too high a drug product concentration may lead to low dose volume and therefore potentially poor distribution throughout the brain. The instant applicant has determined that dose volume limits and target concentration need to be carefully considered to achieve optimal distribution throughout the brain while minimizing the risk of disrupting the CSF pressure balance. In addition, endotoxin levels must be carefully managed and controlled to extremely low levels in both drug product and device components to enable dosing of pediatric patients in compliance with USP, Chapter <85>.

Table 1 below shows the estimated brain weight range, total CSF volume range, and minimum subject body weight provided by age:

TABLE 1

| | | Brain Weight | CSF (cc) | Minimum subject Body |
|---|---|---|---|---|
| Patient Age | | (g)^{a} | range^{b} | Weight (kg) |
| 0 | | 300-500 | 20-50 | 2.4 |
| 6 | months | 500-700 | 20-90 | 5.7 |
| 1 | year | 700-900 | 60-90 | 7.8 |
| 2 | year | 800-1100 | 75-150 | 9.9 |
| 4 | year | 900-1300 | 100-150 | 12.8 |
| 6-9 | year | 1000-1300 | 100-150 | 14.3 |
| 10-18 | year | 1000-1400 | 120-170 | 23.9 |

^{a}Estimates for range of subject brain weight determined by 3rd and 97th percentile of (Peterson et al., J Neurosurg Pediatr. 2021 Jul. 9; 28(4): 458-468.)
^{b}Estimates for CSF volume were determined by Thwaites et al. J Infect. 2009 September; 59(3): 167-87. [symbol]
dy weight based on CDC guidelines [symbol]
indicates data missing or illegible when filed

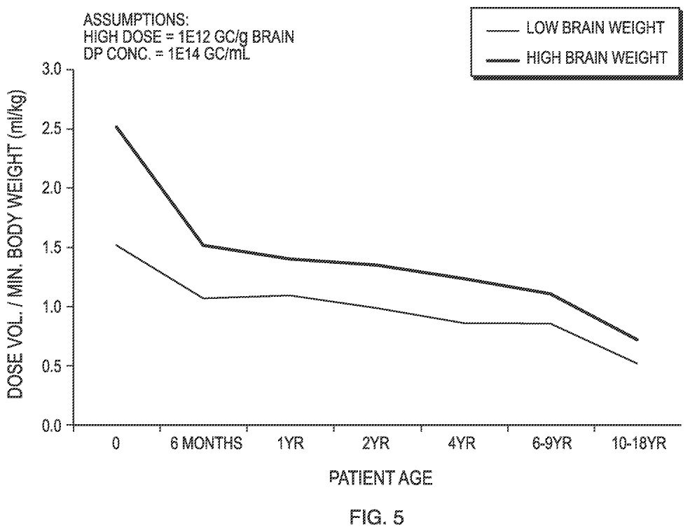

FIG. 5 shows the estimated drug product volume (dose volume) per minimum subject weight with age. Dosing in pediatric patients requires tight control over titer, impurities, and endotoxin levels. Table 2 below illustrates the challenge faced with the pediatric patients in terms of age (0-6 months), lowest brain weight range (300-700 g) and minimum body weight, as depicted in Table 1, corresponding to low brain weight and low CSF volume that place strict constraints on volumes of drug product that can be administered, and where a high drug product concentration is important to allow for efficacious treatment. As demonstrated in the Table 2, low concentration drug products (1×10¹⁴ GC/ml) may require greater than 20% CSF volume of drug product to be administered in the youngest patients and patients with small CSF volume and/or brain weight.

TABLE 2
Effect of Drug Product Concentration on Dose Volume as
% CSF at a target dose of 1 × 10¹² GC/g brain.
% Dose volume in patients with Low CSF Volume
Target dose ~1 × 10¹² GC/g brain
Dosage range 0.8-1.2 × 10¹² GC/g brain (capped
at 1.2 × 10¹² GC/g brain)

| | | Brain | Formulation Concentration (GC/ml) | | | | | | | | | | |
|---|---|---|---|---|---|---|---|---|---|---|---|---|---|
| Approx. | | Weight | Low | | | | Med. | | | High | | | |
| Ages | | (g) | (1 × 10¹⁴) | | | | (1.5 × 10¹⁴) | | | (2 × 10¹⁴) | | | |
| <=6 | mo | 300- | 20% | 18% | 17% | 14% | 13% | 13% | 11% | 11% | 11% | 10% | 10% |
| | | 350 | | | | | | | | | | | |
| | | 351- | 21% | 20% | 18% | 17% | 16% | 15% | 14% | 13% | 11% | 11% | 11% |
| | | 400 | | | | | | | | | | | |
| | | 400- | 24% | 21% | 20% | 18% | 17% | 15% | 15% | 14% | 13% | 12% | 12% |
| | | 450 | | | | | | | | | | | |
| | | 451- | 23% | 21% | 18% | 18% | 16% | 14% | 14% | 13% | 12% | 11% | 11% |
| | | 500 | | | | | | | | | | | |
| | | 501- | 24% | 21% | 20% | 18% | 16% | 16% | 14% | 13% | 13% | 12% | 12% |
| | | 600 | | | | | | | | | | | |
| | | 601- | 22% | 19% | 19% | 16% | 16% | 15% | 12% | 12% | 12% | 11% | 11% |
| | | 700 | | | | | | | | | | | |
| 1-2 | yr | 701- | 20% | 18% | 15% | 15% | 15% | 13% | 12% | 12% | 10% | 10% | 10% |
| | | 800 | | | | | | | | | | | |
| | | 801- | 19% | 16% | 16% | 14% | 12% | 12% | 12% | 10% | 10% | 10% | 9% |
| | | 900 | | | | | | | | | | | |
| | | 901- | 16% | 15% | 13% | 13% | 12% | 10% | 10% | 10% | 10% | 9% | 8% |
| | | 1000 | | | | | | | | | | | |
| 2-4 | yr | 1001- | 14% | 14% | 12% | 11% | 11% | 11% | 10% | 8% | 8% | 8% | 8% |
| | | 1100 | | | | | | | | | | | |
| | | 1101- | 14% | 12% | 12% | 11% | 9% | 9% | 9% | 8% | 7% | 7% | 7% |
| | | 1200 | | | | | | | | | | | |
| 4-9 | yr | 1201- | 12% | 11% | 10% | 9% | 9% | 8% | 8% | 7% | 7% | 6% | 6% |
| | | 1300 | | | | | | | | | | | |
| 9-18 | yr | 1301- | 11% | 10% | 9% | 8% | 8% | 7% | 7% | 6% | 6% | 5% | 5% |
| | | 1400 | | | | | | | | | | | |

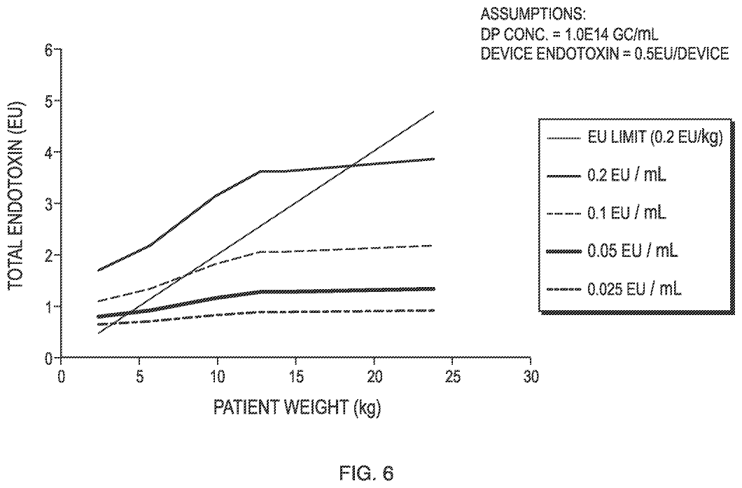

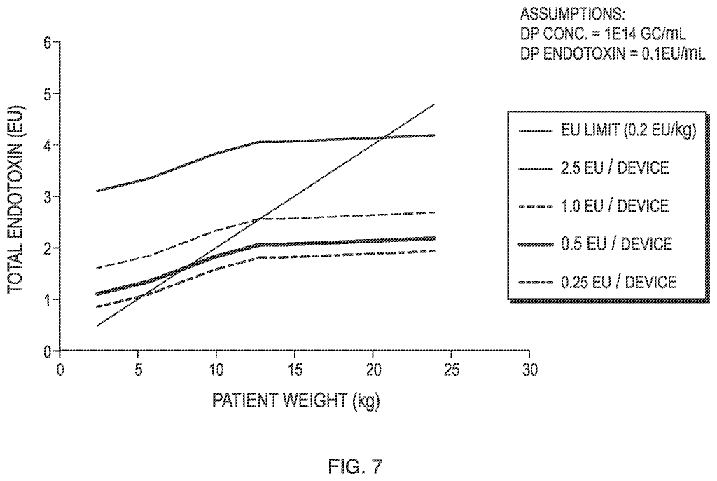

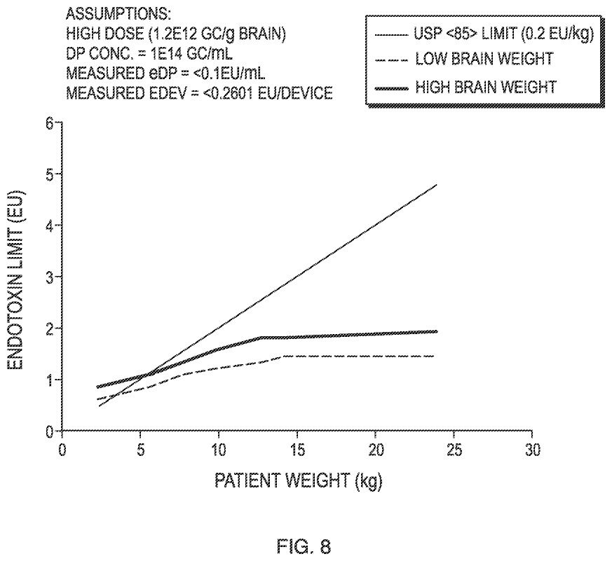

FIG. 6 and FIG. 7 show the effect of drug product and device contribution to the endotoxin risk, respectively. FIG. 8 shows the application of the measured drug product and device components and their contribution to endotoxin, thus illustrating the challenge associated with treating patients with very low body weights. Through optimization of dose concentration targets, tight control of the concentration range, selection of low endotoxin raw materials for the manufacturing process, drug product preparation, and administration device components, the inventors have created a high rAAV concentration formulation that has acceptably low endotoxin levels, therefore allowing for IT, ICV, and/or ICM delivery of CNS-targeting AAV gene therapy products for pediatric patients, e.g., infants, having a very low body weight.

### Example 2

The following example describes the development and evaluation of a mathematical framework for formulating a low endotoxin dose preparation of a drug product (an rAAV-comprising pharmaceutical formulation), taking into consideration endotoxin contributions from the drug product, the dose preparation and administration components used for intrathecal delivery of the drug product.

### Framework for Establishing Drug Product Endotoxin Limits

Described below is a mathematical framework that was developed for use in establishing drug product endotoxin limits, taking into consideration variables including the threshold pyrogenic dose for CSF-administered drugs, patient attributes, preparation and administration component endotoxin levels, and the rAAV drug product, as depicted in Table 3 below.

TABLE 3
Variables for drug product and injection device endotoxin calculation

| Variable | Description | Unit |
|---|---|---|
| C_{UX055} | Concentration of rAAV GC in Drug Product per milliliter | GC/mL |
| D_{brain} | Dose (GCr_{AAV}/ m_{Brain}); high dose = 1 × 10¹² GC/g brain | GC/g of brain |
| e_{DP} | Endotoxin Concentration of Drug Product | EU/mL |
| E_{Dev} | Total Endotoxin Contribution of the Injection Device | EU |
| E_{DP} | Total Endotoxin Contribution of Drug Product | EU |
| Eₗᵢₘ | Total Endotoxin Patient Specific Endotoxin Limit | EU |
| K | 0.2 EU/kg body weight for Intrathecal Bolus Dose (<1 hr) | EU/kg body weight |
| | (USP<85>) | |
| m_{brain} | Brain mass | g of brain |
| m_{body} | Body weight | kg |
| M_{DP} | Dose of drug in volume rAAV drug product/kg body | mL of product/kg body weight |
| | weight | |
| V_{DP} | Volume of Drug Product | mL |

Described below is a basic formula for determining the total endotoxin in the final formulation of the drug product considering the acceptable level of endotoxin for intrathecal delivery at <0.2 EU/kg body weight/hr, as required by the pharmacological standard of Chapter <85> of the United States Pharmacopeia (USP). For a single bolus dose of drug administered in less than 1 hour the time component can be dropped.

$$\frac{Total Endotoxin}{{m}_{b o d y}} < 0.2 EU / kg \qquad Equation . 1$$

Further, the total endotoxin a patient may be exposed to is the sum of contributions from the drug product (E_{DP}) and the administration components (E_{dev}). Incorporating E_{DP} and E_{dev}, Equation. 1 can be re-written as:

$$\frac{{E}_{D P} + {E}_{D e v}}{{m}_{b o d y}} < 0.2 EU / kg \qquad Equation . 2$$

The endotoxin in the drug product, E_{DP}, is the concentration of endotoxin in drug product multiplied by the dose volume (V_{DP}):

$${E}_{D P} = {V}_{D P} * {e}_{D P} \qquad Equation . 3$$

The dose volume (V_{DP}) is the product of the intended dose (D_{brain}) multiplied by the patient's brain mass (m_{brain}) divided by the concentration of rAAV in the drug product (C_{rAAV}):

$${V}_{D P} = \frac{{D}_{b r a i n} * {m}_{B r a i n}}{CrAAV} \qquad Equation . 4$$

Ultimately, a drug product endotoxin specification must be set, accounting for endotoxin contributions from the administration components and the worst-case dosing conditions. To determine the appropriate drug product specification limit, Equation. 2 was rearranged as:

$${E}_{D P} < 0.2 \frac{E U}{kg} \cdot {m}_{b o d y} - {E}_{D e v} \qquad Equation . 5$$

Equation. 3 and Equation. 5 were combined to calculate the endotoxin concentration in the drug product (instead of total endotoxin):

$${e}_{D P} < \frac{0.2 \frac{EU}{kg} \cdot {m}_{b o d y} - {E}_{D e v}}{{V}_{D P}} \qquad Equation . 6$$

Finally, Equation. 4 and Equation. 6 were combined to incorporate the additional dosing factors:

$${e}_{D P} < \frac{0.2 \frac{EU}{kg} \cdot {m}_{b o d y} - {E}_{D e v}}{{C}_{rAAV}} \qquad Equation . 7$$

In Equation. 7, the following conditions that lead to the most stringent endotoxin requirements, where eDP is minimized, were identified:
High contribution from administration components (E_{dev}).
High brain/body weight ratios (m_{brain}/m_{body}).
High doses (D_{brain})
Low DP concentrations (C_{rAAV})

### Example 3

Described below is a description of the process of setting the rAAV endotoxin specification limit based on the mathematical framework described in Example 2.

Endotoxin Contribution from the Dose Preparation and Administration Components

The endotoxin contribution from the dose preparation and administration components, referred to as E_{dev} in the calculations above, was measured. The study described herein found that, in the worst-case scenario, using the highest dosing volume and maximum number of components used to prepare and administer the product, a set of dose preparation and administration components would contribute <0.32 EU to the patient's endotoxin exposure.

### Endotoxin and Brain/Body Weight Ratio

The rAAV dose was based on brain weight (m_{brain}) and the intrathecal endotoxin limit (EU) was based on minimum patient body weight (m_{body}), such that patients with the highest brain to body weight ratio (typically, youngest and/or smallest patients) have the highest risk of endotoxin exposure. Table 4 below summarizes determination of the range of endotoxin limit required (e_{DP} or K/M_{DP}, measured as EU/ml) shown in the right most column of the table, calculated using equation 7 described in Example 2, based on the ranges of patient m_{brain}, minimum m_{body} for different age groups, intrathecal endotoxin limit (EU) corresponding to the minimum m_{body} based on tolerable endotoxin limit of 0.2 EU/kg as per USP<85>, ranges of rAAV drug concentration (GC/ml), required drug dose (total GC), and total drug volume of the rAAV dose (V_{DP}). As described in Table 4, in younger subjects, with higher brain to body weight ratios, the endotoxin limit is more stringent, and endotoxin specifications must be set with the youngest intended patients in mind.

TABLE 4
The rAAV drug product endotoxin limit determination (K/M_{DP}) for High dose (1.2 ×
10¹² GC/g brain, capped at total 1.2 × 10¹⁵ GC, assuming device endotoxin contribution of 0.32 EU)

| | | | | Intrathecal | | | | | |
|---|---|---|---|---|---|---|---|---|---|
| | | | | Endotoxin | | | | | |
| | | | | Limit | | | | | |
| | | | | (EU)* | | | | | |
| | | Brain | Min | per | | | | | K/M_{DP} |
| | | Weight, | Body | USP<85> | rAAV drug | | V_{DP} Drug | Estim. | Endotoxin |
| | | m_{brain} | Wt, | Bodyweight | concentration | Req. total rAAV | Product | Device | limit req (e_{DP}). |
| Patient | | (cc, g) | m_{body} | (kg) * | (C_{DP})(GC/mL) | Dose (total GC) | Volume (mL) | Endotoxin | (EU/mL) |
| Age | | Low-High | (kg) | (0.2 Eu/kg) | Low-High | Low-High | Low-High | (EU)** | High-Low |
| 0 | yr | 300-500 | 2.4 | 0.48 | 1.0E+14-2.0E+14 | 3.6E+14-6.0E+14 | 1.8-6.0 | 0.32 | 0.09-0.03 |
| 6 | months | 500-700 | 5.7 | 1.14 | 1.0E+14-2.0E+14 | 6.0E+14-8.4E+14 | 3.0-8.4 | 0.32 | 0.27-0.10 |
| 1 | yr | 700-900 | 7.8 | 1.56 | 1.0E+14-2.0E+14 | 8.4E+14-1.1E+15 | 4.2-10.8 | 0.32 | 0.30-0.11 |
| 2 | yr | 800-1100 | 9.9 | 1.98 | 1.0E+14-2.0E+14 | 9.6E+14-1.2E+15 | 4.8-12.0 | 0.32 | 0.35-0.14 |
| 4 | yr | 900-1300 | 12.8 | 2.56 | 1.0E+14-2.0E+14 | 1.1E+15-1.2E+15 | 5.4-12.0 | 0.32 | 0.41-0.19 |
| 6-9 | yr | 1000-1300 | 14.3 | 2.86 | 1.0E+14-2.0E+14 | 1.2E+15-1.2E+15 | 6.0-12.0 | 0.32 | 0.42-0.2 |
| 10-18 | yr | 1000-1400 | 23.9 | 4.78 | 1.0E+14-2.0E+14 | 1.2E+15-1.2E+15 | 6.0-12.0 | 0.32 | 0.74-0.3 |

The examples described herein show that the mathematical framework disclosed herein can be used to calculate the range of tolerable endotoxin limit of a rAAV drug product for specific drug dose and drug concentration based on a predetermined fixed endotoxin contribution from the dose preparation and administration components, patient brain weight and body weight, to attain the pharmacologically tolerable endotoxin limit of 0.2 EU/kg.

### INCORPORATION BY REFERENCE

The entire disclosure of each of the patent documents and scientific articles referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Various structural elements of the different embodiments and various disclosed method steps may be utilized in various combinations and permutations, and all such variants are to be considered forms of the disclosure. Scope of the disclosure is thus indicated by the appended claims rather than by the foregoing description, and all changes that come within the meaning and range of equivalency of the claims are intended to be embraced therein.

What is claimed is:

1. A pharmaceutical formulation for administration to the central nervous system (CNS) of a subject, the pharmaceutical formulation comprising a recombinant adeno-associated virus (rAAV) at a concentration of at least 5×10¹³ genome copies (GC)/mL and having an endotoxin concentration of less than or equal to 0.2 endotoxin unit (EU)/mL.

2. The pharmaceutical formulation of claim 1, wherein the pharmaceutical formulation comprises an rAAV at a concentration of at least 5×10¹³ GC/mL, at least 6×10¹³ GC/mL, at least 7×10¹³ GC/mL, at least 8×10¹³ GC/mL, at least 9×10¹³ GC/mL, at least 1×10¹⁴ GC/mL, at least 1.5×10¹⁴ GC/mL, at least 2×10¹⁴ GC/mL, at least 3×10¹⁴ GC/mL, at least 4×10¹⁴ GC/mL, at least 5×10¹⁴ GC/mL, at least 6×10¹⁴ GC/mL, at least 7×10¹⁴ GC/mL, at least 8×10¹⁴ GC/mL, at least 9×10¹⁴ GC/mL, at least 1×10¹⁵ GC/mL, at least 3×10¹⁵ GC/mL, or at least 5×10¹⁵ GC/mL.

3. The pharmaceutical formulation of claim 1 or 2, wherein the pharmaceutical formulation has an endotoxin concentration of less than or equal to 0.2 EU/mL, less than or equal to 0.15 EU/mL, less than or equal to 0.1 EU/mL, less than or equal to 0.05 EU/mL, less than or equal to 0.03 EU/mL, or less than or equal to 0.01 EU/mL.

4. The pharmaceutical formulation of any one of claims 1-3, wherein the pharmaceutical formulation comprises rAAV at a concentration of at least 1×10¹⁴ GC/mL and has an endotoxin concentration of less than or equal to 0.1 EU/mL.

5. The pharmaceutical formulation of any one of claims 1-3, wherein the pharmaceutical formulation comprises rAAV at a concentration of at least 1.5×10¹⁴ GC/mL and has an endotoxin concentration of less than or equal to 0.1 EU/mL.

6. The pharmaceutical formulation of any one of claims 1-5, wherein the pharmaceutical formulation is dosed at 0.1×10¹² GC/g brain to 3×10¹² GC/g brain of the subject.

7. The pharmaceutical formulation of any one of claims 1-6, wherein the pharmaceutical formulation comprises sodium phosphate, sodium chloride (NaCl), sorbitol, poloxamer 188, potassium chloride (KCl), magnesium chloride (MgCl₂), calcium chloride (CaCl₂)), or a combination thereof.

8. The pharmaceutical formulation of claim 7, wherein the pharmaceutical formulation comprises sorbitol at a concentration of at least 1% to at least 20% of the total formulation.

9. The pharmaceutical formulation of claim 7, wherein the pharmaceutical formulation comprises sorbitol at a concentration of at least 5% to at least 10% of the total formulation.

10. The pharmaceutical formulation of any one of claims 1-9, wherein the pharmaceutical formulation comprises: (i) 10 mM sodium phosphate, 135 mM NaCl, 0.27M Sorbitol, 0.001% v/v poloxamer 188, pH 7.2; (ii) 10 mM sodium phosphate, 135 mM NaCl, 0.54M Sorbitol, 0.001% v/v poloxamer 188, pH 7.2; or (iii) 10 mM Tris, 130 mM NaCl, 2 mM KCl, 1 mM MgCl₂, 2 mM CaCl₂), 0.27M Sorbitol, 0.001% v/v poloxamer 188, pH 6.8.

11. The pharmaceutical formulation of claim 10, wherein the pharmaceutical formulation comprises 10 mM sodium phosphate, 135 mM NaCl, 0.27M Sorbitol, 0.001% v/v poloxamer 188, pH 7.2.

12. The pharmaceutical formulation of any one of claims 1-11, wherein the subject is a pediatric subject.

13. The pharmaceutical formulation of any one of claims 1-12 for use in treating a CNS disorder in the subject.

14. The pharmaceutical formulation of claim 13, wherein the CNS disorder is selected from CDKL5 deficiency disorder (CDD), Angelman syndrome, Batten disease, Krabbe disease, Parkinson's disease, Alzheimer's disease, Spinal Muscular Atrophy (SMA) Types I, II, III, or IV, X-linked Myotubular Myopathy, Friedrich's Ataxia, Canavan's disease, Amyotrophic Lateral Sclerosis (ALS), Adrenoleukodystrophy, Huntington disease, Rett syndrome, or Spinocerebellar ataxia.

15. The pharmaceutical formulation of claim 14, wherein the CNS disorder is CDD and the rAAV comprises a nucleic acid sequence encoding a CDKL5 polypeptide.

16. A method of treating a CNS disorder in a subject comprising administering to the CNS of the subject the pharmaceutical formulation of any one of claims 1-11.

17. The method of claim 16, wherein the subject is a pediatric subject.

18. The method of claim 16 or 17, wherein the CNS disorder is CDKL5 deficiency disorder (CDD), Angelman syndrome, Batten disease, Krabbe disease, Parkinson's disease, Alzheimer's disease, Spinal Muscular Atrophy (SMA) Types I, II, III, or IV, X-linked Myotubular Myopathy, Friedrich's Ataxia, Canavan's disease, Amyotrophic Lateral Sclerosis (ALS), Adrenoleukodystrophy, Huntington disease, Rett syndrome, or Spinocerebellar ataxia.

19. The method of claim 18, wherein the CNS disorder is CDD and the rAAV comprises a nucleic acid sequence encoding a CDKL5 polypeptide.

20. The method of any one of claims 16-19, wherein the pharmaceutical formulation is administered intrathecally, intracerebroventricularly, or via an intracisternal magna route.

21. The method of claim 20, wherein the administration is a bolus injection.

22. The method of claim 20, wherein the administration is via continuous infusion.

23. The method of claim 20, wherein the pharmaceutical formulation is administered at a dose of 0.5 mL to 12 mL.

24. The method of claim 23, wherein the pharmaceutical formulation is administered at a dose of 2 mL to 8 mL.
